(19) United States
(12) Patent Application Publication
(10) Pub. No.: US 2012/0058499 A1
(43) Pub. Date: Mar. 8, 2012

(54) Bioluminescent Detection of Protease Activity

(76) Inventors: Steven J. Orcutt, Audubon, PA (US); Yan Yilin, (US); Jian Wu, Frazer, PA (US); James Strickler, Havertown, PA (US); Tauseef R. Butt, Malvern, PA (US)

(21) Appl. No.: 13/157,734

(22) Filed: Jun. 10, 2011

Related U.S. Application Data
(60) Provisional application No. 61/353,956, filed on Jun. 11, 2010.

(51) Int. Cl. C12Q 1/66; C07K 2/00

(52) U.S. Cl. 435/8; 530/300

(57) ABSTRACT

Methods for bioluminescent detection of the activity of proteolytic enzymes including ubiquitin (Ub) and ubiquitin-like (Ubl) proteolytic enzymes are disclosed.

This application claims priority under 35 U.S.C. §119(e) to U.S. Provisional Patent Application No. 61/353,956, filed on Jun. 11, 2010. The foregoing application is incorporated by reference herein.

### FIELD OF THE INVENTION

The present invention relates to the field of detecting the activity of proteolytic enzymes such as ubiquitin (Ub) and ubiquitin-like (Ubl) proteolytic enzymes. More specifically, the present invention provides materials and methods for improved sensitivity in the bioluminescent (e.g., luciferase technology) detection of proteolytic enzymes by use of protein substrates based on Ub and Ubl molecules.

### BACKGROUND OF THE INVENTION

Several publications and patent documents are cited throughout the specification in order to describe the state of the art to which this invention pertains. Full citations of these references can be found throughout the specification. Each of these citations is incorporated herein by reference as though set forth in full.

Proteases (also referred to as proteinases, peptidases or proteolytic enzymes) are enzymes which hydrolyze bonds within polypeptides/proteins (e.g., isopeptide bonds and peptide bonds) and constitute a large and important group of enzymes involved in diverse physiological processes such as blood coagulation, inflammation, reproduction, fibrinolysis, cellular apoptosis, and the immune response. Numerous disease states are caused by, and can be characterized by, observed alterations in the activity of specific proteases and their inhibitors. The ability to measure protease activity in research, clinically or otherwise, is significant to the investigation, treatment and management of disease states. Proteases include, without limitations, serine proteases, threonine proteases, cysteine proteases, aspartate proteases, metalloproteases, and glutamic acid proteases. A database of known proteases is available at merops.sanger.ac.uk (Rawlings et al., 2004, Nucleic Acids Res., 32 (Database issue): D160-D164).

Ubiquitin (Ub) and ubiquitin-like proteins (Ubls) have been described in the literature (Jentsch & Pyrowolakis, 2000, Trends Cell Biol., 10:335-42; Yeh et al., 2000, Gene, 248:1-14; Larsen & Wang, 2002, J. Proteome Res., 1:411-9; Schanz et al., 2003, Trends Biochem. Sci., 28:321-238). Ubls include such proteins as SUMO-1 (small ubiquitin-related modifier-1; also known as Sentrin, SMT3, PIC1, GMP1 and UBL1), SUMO-2, SUMO-3, ISG-15 (interferon stimulated gene 15; also known as UCRP (ubiquitin cross-reactive protein)), and NEDD-8 (neural precursor cell expressed developmentally down-regulated 8; also known as RUB1 (related to ubiquitin 1)). Regulation of cellular processes through the modification of target proteins by Ub/Ubls has been an area of intense research since the discovery that proteins modified with one ubiquitin (monoubiquitinated), more than one ubiquitin molecule at multiple sites (multiubiquitinated), or more than one ubiquitin in polymer chain form (polyubiquitinated) have an altered cellular fate from those proteins not having an attached ubiquitin (Pickart, C. M., Mechanisms Underlying Ubiquitin, 2001, Annu. Rev. Biochem. 70, 503-533). Ubiquitin is conjugated, either in monomer or polymer form, to the target protein at the ε-NH₂ of lysine amino acids by a series of enzymatic proteins (E1, E2, E3) that co-ordinate ubiquitin ligation.

Isopeptidases constitute a family of deconjugating enzymes that are responsible for the proteolytic removal of Ub/Ubls from proteins existing as Ub/Ubls conjugates. Removal of ubiquitin(s) from target proteins is carried out by deubiquitinases (also known as deubiquitylating or deubiquitinating enzymes) (DUBs) (Wilkinson, 2000, Semin. Cell Dev. Biol., 11:141-148; Ciechanover, 2003, Biochem. Soc. Trans., 31:474-481). Analogous machinery exists to regulate proteins through conjugation of other Ubls, such as SUMO. Sumoylation of cellular proteins has been proposed to regulate nuclear transport, signal transduction, stress response, and cell cycle progression (Kretz-Remy & Tanguay, 1999, Biochem. Cell. Biol., 77:299-309). Deconjugating or removal of Ubls such as SUMO is carried out by isopeptidases called deSUMOylating proteases (SENPs); deconjugating or removal of Ubls such as NEDD-8 is carried out by isopeptidases called deneddylating proteases or deneddylases (NEDPs).

The regulation of intracellular protein levels of Ub/Ubl conjugation is associated with particular disease states, and observed isopeptidase activity can be directly related to these disease states. Therefore, it is highly desirous to be able to detect isopeptidase activity with increased sensitivity, in both an in vitro setting and in the cellular milieu.

A number of assay formats have been used for the measurement of isopeptidase/DUB activity. The first of these is the use of Ub/Ubls conjugated via a peptide bond to a fluorescent molecule, such as amino-methylcoumarin (AMC) or rhodamine110, that allows for measurement of increased fluorescence intensity as the fluorophore is liberated from the Ub/Ubl molecule (Hassiepen et al., 2007, Analyt. Biochem., 371:201-207). The use of fluorophores linked to enzymatic substrates for the detection of protease enzymes is described, for example, in U.S. Pat. No. 4,336,186. The primary disadvantage of this format is that intrinsic fluorescence of the conjugated molecule is observed and can often lead to a background signal that precludes achieving sufficient signal to background (S/B) levels in the assay. In addition, fluorescent artifacts (such as auto-fluorescence and spectral quenching) can hinder the drug discovery process with small organic molecules.

A second format, the Ub/Ubl CHOP reporter system (LifeSensors, Inc. (Malvern, Pa.); see e.g., www.lifesensors.com/search-results.php?q=CHOP&filter=all&page=2#mainContent), improves upon some limitations of the Ub-fluorophore conjugate format (Nicholson et al., 2008, Protein Science, 17:1-9; US Patent Application Publication No. 2006-0040335A1). Ub/Ubl is conjugated via a peptide bond to an inactive precursor form of a reporter enzyme. The reporter enzyme requires a free N-terminus, achieved through isopeptidase cleavage, to become active. Upon DUB cleavage, the activity of the resultant reporter enzyme against its own substrate is monitored. As the measured signal is proportional to the amount of generated reporter enzyme, one can use this signal to deduce isopeptidase activity. This so-called “coupled” assay in effect amplifies the isopeptidase activity, allowing for screening at lower concentrations of protease.

A third format, the LanthScreen™ Deubiquitination Assay (Invitrogen, Carlsbad, Calif.) is a time-resolved fluorescence resonance energy transfer (TR-FRET) assay. In this assay, Ub/Ubl is conjugated at its C-terminus to a fluorescent donor organic molecule and at its N-terminus to yellow fluorescent protein (YFP). Cleavage by the isopeptidase releases the fluorescent donor from the molecule, and is associated with a decrease in the observed signal (US Patent Application Publication No. 2007-0264678 A1). A major disadvantage of this assay format is the observation that perturbations at the N-terminus of ubiquitin impact ubiquitin structure/function.

A fourth format uses bioluminescence or luminescence based upon luciferase technology to monitor isopeptidase cleavage, using a substrate composed of the five C-terminal amino acids of ubiquitin conjugated to an amino-luciferin molecule (DUB-Glo™ (Promega, Inc., Madison, Wis.)). This substrate is cleaved by a wide range of Ub/Ubl-specific proteases to varying degrees. This peptide, however, is a very poor substrate for ubiquitin and Ubl processing enzymes with respect to both affinity (defined as the ability of the substrate to bind to the enzyme prior to catalysis) and catalytic rate (defined as peptide bond cleavage resulting in free luciferin). Kinetic studies with isopeptidase T demonstrated that the relative catalytic efficiency (kcat/Km) for the cleavage of a full-length ubiquitin AMC derivative was ˜8000-fold higher compared to an AMC derivative of the Z (carboxybenzyl)-Leu-Arg-Gly-Gly (SEQ ID NO: 29) peptide (Dang et al., 1998, Biochemistry, 37:1868-79). Moreover, the kcat/Km for UCHL3 activity towards full length ubiquitin-AMC was 178×10⁶ M⁻¹ s⁻¹, compared to the published value of 2.4 M⁻¹ s⁻¹ for Z-Leu-Arg-Gly-Gly (SEQ ID NO: 29)-AFC (7-amino-4-trifluoromethylcoumarin) (Drag et al., 2008, Biochem. J., 415:367-375).

Luciferase technology has also been used in bioluminescent assays with short peptide substrates for the detection of caspases, trypsin and tryptase (WO 2003/066611; US Patent Application Publication No. 2006-0121546 A1; U.S. Pat. No. 7,148,030; U.S. Pat. No. 7,384,758; U.S. Pat. No. 7,666,987). Prior to the application of luciferase technology, the activities of trypsin-, tryptase-, and caspase-like enzymes were classically monitored by their cleavage of short peptidyl substrates conjugated to compounds that, upon release, would increase in spectral absorbance (para-nitroaniline) or fluorescence (e.g., AMC and rhodamine110).

The mechanism by which certain Ub/Ubl-specific proteases recognize and cleave their cognate Ub/Ubl substrate (“specificity”) is not uniform. At the gene level, ubiquitin is encoded as a head-to-tail linked poly-ubiquitin (6-15 units of monomer Ub arranged in a head-to-tail fashion). Ubiquitin is also encoded as monomers linked to a C-terminal extension, such as a Ub-ribosomal fusion protein. In order for ubiquitin to enter the ubiquitinylation pathway and for conjugation of the C-terminus of ubiquitin to target proteins, linear poly-ubiquitin or ubiquitin carboxyl extension proteins must be cleaved by DUBs to form mature ubiquitins. Among the ˜100 DUBs encoded by the human genome, only a subset of DUBs are responsible for the generation of free ubiquitin to enter the ubiquitin pathway. Ubls, such as SUMO, are also encoded at the gene level in precursor form. Thus, nature has designed certain DUBs that recognize Ub and Ubl C-terminal peptide extensions as substrates. In these cases, specificity is thought to be determined by discrete interactions between the protease and the amino acid residues in and around the active site. This assumption of specificity has led to the wide spread use of Ub/Ubl conjugates that have a small adjunct (typically fluorescent in nature) linked to a C-terminus peptide. While these conjugates are cleaved to a measureable degree by some Ub/Ubl specific proteases (such as UCHL3 (ubiquitin carboxyl-terminal hydrolase isozyme L3), SENP2 (sentrin-specific protease 2), PLpro (papain-like protease)), other Ub/Ubl-specific proteases exhibit no detectable activity towards these reporter molecules (such as Otubain2 (Otub2), AMSH (associated molecule with the SH3 domain of STAM), JosD1 (Josephin-1)). The activity of enzymes such as Otub2, AMSH, and JosD1 can be detected by the cumbersome and time consuming monitoring of polyubiquitin degradation by immunoblotting. Presumably, the poor reactivity of Ub/Ubl C-terminal adducts with certain proteases is related to specificity requirements beyond discrete interactions with amino acids surrounding the bond to be cleaved. Considering that the majority of known Ub/Ubl-specific proteases (100+) have not been adequately characterized with respect to relative activity or specificity, there exists a true need for novel reagents for the characterization of these isopeptidases. It is also desirable to provide an assay employing a Ub or Ubl attached to an adduct that provides for the highly sensitive detection of protease activity.

There remains an unmet need to provide assays of specific protease activity that are of greater sensitivity, higher specificity, enhanced catalytic efficiency, and that have higher signal to background ratios (S/B) and increased lower limits of detection than existing assays formats.

### SUMMARY OF THE INVENTION

The present invention provides methods for detecting protease activity in a sample comprising contacting the sample with (i) a protease substrate that comprises (a) a first moiety comprising at least one ubiquitin (Ub) or a ubiquitin-like protein (Ubl), said first moiety comprising at its C-terminus a cleavage site for the protease, and (b) a second moiety comprising a luciferase substrate, wherein the first moiety is covalently linked at its C-terminus to the second moiety (e.g., via an amide linkage), and (ii) luciferase, wherein the isopeptidase cleaves the protease substrate at the C-terminal end of the first moiety, thereby generating free luciferase substrate, and detecting luminescence in the sample, wherein luminescence is indicative of protease activity.

Proteases include, without limitations, serine proteases, threonine proteases, cysteine proteases, aspartate proteases, metalloproteases, glutamic acid proteases, caspases, trypsins, tryptases, isopeptidases, cathepsins, chymotrypsin, secretases (e.g., β-secretases), and ubiquitin (Ub) and ubiquitin-like (Ubl) proteolytic enzymes.

In some embodiments, the protease is a ubiquitin (Ub) and ubiquitin-like (Ubl) proteolytic enzyme selected from the group consisting of UCHL3, USP2core, USP7, USP8, USP34, Otub2, JosD1, JosD2, AMSH, Ataxin3, Ataxin3-like, UCHL5, USP20, USP14, ULP1, Ulp2, SENP1, SENP2, A20 and SENP5.

In some embodiments, the present invention provides methods for detecting proteolytic enzyme activity in a sample comprising contacting the sample with an (i) proteolytic enzyme substrate that comprises (a) a first moiety comprising at least one ubiquitin (Ub) or a ubiquitin-like protein (Ubl), and (b) a second moiety comprising a luciferase substrate, wherein the first moiety is covalently linked at its C-terminus to the second moiety via an amide linkage, and (ii) luciferase, wherein the proteloytic enzyme cleaves the substrate (e.g., at the C-terminal end of the first moiety), thereby generating free luciferase substrate, and detecting luminescence in the sample, wherein luminescence is indicative of proteolytic enzyme activity.

In some embodiments, the luminescence has a signal to background ratio (S/B) about 10- to about 1000-fold greater than the S/B for a corresponding assay wherein the first moiety is a C-terminal peptide of Ub or a Ubl and/or the second moiety is a fluorophore. In some embodiments, the luciferase substrate is luciferin or coelenterazine. In some embodiments, the luciferase comes from a beetle species such as Photinus pyralis. In some embodiments, the luciferase comes from a bioluminescent aquatic organism such as the jellyfish Aequorea victoria, the sea pansy Renilla reniformis, or the copepod (small crustacean) Gaussia princeps. In some embodiments, the Ubl is selected from the group consisting of small ubiquitin like-modifier-1 (SUMO), SUMO-2, SUMO-3, ISG-15, NEDD-8, HUB1, ISG-15, APG12, URM1, and APG8.

The present invention also provides protease substrates comprising a first moiety comprising at least one ubiquitin (Ub) or a ubiquitin-like protein (Ubl), said first moiety comprising at its C-terminus a cleavage site for the protease, and a second moiety comprising a luciferase substrate, wherein the first moiety is covalently linked at its C-terminus to the second moiety via an amide linkage. In particular embodiments, the first moiety comprises at its C-terminus, an amino acid sequence that confers selective cleavage for a protease.

The present invention also provides methods for screening for agents capable of modulating the activity of a protease, comprising, in the presence and the absence of a test agent, (A) contacting the protease with (i) a protease substrate comprising (a) a first moiety comprising at least one ubiquitin (Ub) or a ubiquitin-like protein (Ubl), said first moiety comprising at its C-terminus a cleavage site for the protease, and (b) a second moiety comprising a luciferase substrate, wherein the first moiety is covalently linked at its C-terminus to the second moiety via an amide linkage, and (ii) luciferase, wherein the protease cleaves the protease substrate at the C-terminal end of the first moiety, thereby generating free luciferase substrate, and (B) detecting luminescence in the sample, wherein luminescence is indicative of the protease activity, wherein a difference in the level of luminescence in the presence of the test agent as compared to the absence of the test agent is indicative of an agent that is capable of modulating the activity of the protease.

The present invention also provides methods for diagnosing a disease or condition associated with a protease, comprising (A) contacting a sample from a subject suspected of having the disease or condition with (i) a protease substrate comprising (a) a first moiety comprising ubiquitin (Ub) or a ubiquitin-like protein (Ubl), said first moiety comprising at its C-terminus a cleavage site for the protease, and (b) a second moiety comprising a luciferase substrate, wherein the first moiety is covalently linked at its C-terminus to the second moiety via an amide linkage, and (ii) luciferase, wherein the protease cleaves the protease substrate at the C-terminal end of the first moiety, thereby generating free luciferase substrate, and (B) detecting luminescence in the sample, wherein luminescence is indicative of the protease activity.

The present invention also provides kits for detecting protease activity, comprising a protease substrate of the invention. Kits of the present invention can optionally comprise a luciferase and/or instruction materials.

### BRIEF DESCRIPTION OF THE DRAWINGS

FIG. 1 is a schematic drawing of the preparation of Ub(1-76)-aminoluciferin (also referred to herein as Ub-amino-luciferin). Ubiquitin(1-75)-MESNa (Ub-MESNa; a thioester derivative of ubiquitin) is conjugated to glycyl-D-amino-luciferin to yield full-length ubiquitin (ubiquitin(1-76)) that is covalently linked at its C-terminus, via an amide linkage, to luciferin.

FIG. 2 is a graph plotting signal to background (S/B) values on the y-axis against the nanomolar (nM) concentration of deubiquitinase (DUB) on the x-axis, for the activities of four different DUBs (UCHL3, USP7 (ubiquitin specific peptidase 7), USP8, and USP2core (the common catalytic core domain of the two isoforms of USP2, USP2a and USP2b (Nicholson et al., 2008, Protein Science 17:1-9))) on each of two different DUB substrates: ZRLRGG (SEQ ID NO: 1)-LUC (“Peptide-LUC”, also referred to herein as Z-RLRGG(SEQ ID NO: 1)-amino-luciferin) and Ub-LUC (also referred to herein as ubiquitin-amino-luciferin). Values for UCHL3, USP7, USP8, and USP2core enzymes assayed with the Z-RLRGG(SEQ ID NO: 1)-LUC substrate are represented by open squares, triangles, circles and diamonds, respectively. Values for UCHL3, USP7, USP8, and USPcore enzymes assayed with the Ub-Luc substrate are represented by filled squares, triangles, circles and diamonds, respectively.

FIG. 3 is a graph plotting signal to background (S/B) values on the y-axis against the nanomolar (nM) concentration of deubiquitinase (DUB) on the x-axis, for the activities of four different DUBs (UCHL3, USP7, USP8, and USP2core) on each of two different DUB substrates: Ub-LUC (also referred to herein as ubiquitin-amino-luciferin) and Ub-AMC (also referred to herein as ubiquitin-7-amino-4-methylcoumarin). Values for UCHL3, USP7, USP8, and USP2core enzymes assayed with the Ub-LUC substrate are represented by filled squares, triangles, circles and diamonds, respectively. Values for UCHL3, USP7, USP8, and USP2core enzymes assayed with the Ub-AMC substrate are represented by open squares, triangles, circles and diamonds, respectively.

FIG. 4 is a graph plotting signal to background (S/B) values on the y-axis against the nanomolar (nM) concentration of deubiquitinase (DUB) on the x-axis, for the activities of four different DUBs (UCHL3, USP7, USP8, and USP2core) on each of two different DUB substrates: Ub-LUC (also referred to herein as ubiquitin-amino-luciferin) and Ub-Rh110 (“Ub-Rho”, also referred to herein as ubiquitin-rhodamine110). Values for UCHL3, USP7, USP8, and USP2core enzymes assayed with the Ub-LUC substrate are represented by filled squares, triangles, circles and diamonds, respectively. Values for UCHL3, USP7, USP8, and USP2core enzymes assayed with the Ub-Rh110 substrate are represented by open squares, triangles, circles and diamonds, respectively.

FIG. 5 is a bar graph plotting signal to background (S/B) values on the y-axis against each of nine different DUBs (Otub2, JosD1, JosD2, AMSH, Ataxin3, Ataxin3-like, UCHL5, USP20, and USP14) whose activities were assessed using each of four different DUB substrates (ZRLRGG(SEQ ID NO: 1)-LUC, Ub-LUC, Ub-AMC, and Rh110), along the x-axis. S/B values for ZRLRGG(SEQ ID NO: 1)-LUC, Ub-LUC, Ub-AMC, and Rh110 are represented by black, white, light gray, and dark gray bars, respectively.

FIG. 6 is a graph of the signal (in relative luminescence units (RLUs)) to background ratio (S/B) of hSUMO2-amino-luciferin substrate compared to the Z-Arg-Leu-Arg-Gly-Gly (SEQ ID NO: 1)-amino-luciferin substrate with six different isopeptidases (USP34, USP7, USP8, SENP1, SENP2, and SENP6).

FIG. 7 is a graph of the RLUs of the NEDD8-amino-luciferin substrate compared to the Z-Arg-Leu-Arg-Gly-Gly (SEQ ID NO: 1)-amino-luciferin substrate with the isopeptidase DEN 1 over the indicated concentrations.

### DETAILED DESCRIPTION OF THE INVENTION

The present invention provides methods and corresponding reagents for increased sensitivity in the bioluminescent detection of protease activity. In some embodiments, the present invention provides a bioluminescent assay and corresponding reagents for improved sensitivity in the detection of a wide variety of proteases, including, without limitation, caspases, cathepsins, chymotrypsins, secretases, trypsins, tryptases, and isopeptidases. The methods and corresponding reagents are based, in part, on the discovery that attaching a luciferin substrate to the C-terminus of ubiquitin (Ub) or ubiquitin-like proteins (Ubls) provides an improved substrate for proteolytic enzymes that specifically cleave at the C-terminus of Ub or Ubls, including, but not limited to, small ubiquitin like-modifier-1 (SUMO), SUMO-2, SUMO-3, ISG-15, and NEDD-8. In a particular embodiment, the methods and corresponding reagents of the present invention, incorporate the cleavage site of a protease of interest at the C-terminus of a Ub or Ubl protein moiety that is covalently linked at the C-terminus to a luciferase substrate moiety, such that upon action of the protease at its cleavage site, the luciferase substrate is liberated and can be acted upon by a luciferase and detected.

The present invention represents an improvement over existing technologies for the detection of protease, including isopeptidase, activity. The methods and corresponding reagents of the present invention provide greater sensitivity, higher specificity, enhanced catalytic efficiency, and higher signal to background ratios (S/B), allowing for increased lower limits of detection of proteases, including isopeptidases, over existing protease, including isopeptidase, activity detection methodologies.

Herein, it is demonstrated that Ub/Ubl-luciferins provide an unexpectedly superior reagent for the detection of proteolytic activity, particularly for certain DUBs. Indeed, similar molecules with small reporter adjuncts at the C-terminus (e.g., Ub-AMC or Ub-Rho110) have not been shown to be significantly active with DUBS. While not wishing to be bound by any particular theory, it is believed that DUBs recognize and cleave Ub- and Ubl-luciferins with greater affinity due to the structural differences related to other adjuncts (e.g., AMC or Rho110), in addition to benefiting from the amplification nature of the luciferin/luciferase system.

Herein, it is also demonstrated that Ub- and Ubl-luciferins are superior to short peptide-luciferins as substrates for DUBs. While not wishing to be bound by any particular theory, it is believed that the DUBs bind the Ub- and Ubl-luciferins with greater affinity than short peptides. These properties are also applicable to the design of luciferase substrates for other proteases containing cleavage recognition sites at the C-terminus of the Ub/Ubl moiety.

Although not wishing to be bound by any particular theory, short peptides may tumble and fail to efficiently present their cleavage recognition sites to proteases. However, attachment of a protease cleavage recognition sequence (cleavage site) to the C-terminus of a Ub or Ubl protein allows the protease cleavage recognition sequence to be presented to the protease in a much more efficient manner. The C-terminus of Ub or Ubl is normally extended from the compact body of the rest of the Ub or Ubl protein just like a “ball” (compact Ub/Ubl) and a “chain” (Ub/Ubl C-terminus). In the protease substrates of the invention, the “ball” comprises the compact portion of the Ub/Ubl protein and the “chain” comprises the C-terminal protease cleavage site linked to the luciferase substrate. In some embodiments, this new structure is about 10-times longer than short peptide-luciferase substrates. Since the five C-terminal amino acids of Ub/Ubls are not believed to have any significant tertiary structure, and extend like a chain from the compact ball of the rest of the protein, this linear C-terminal structure is believed to be the best site to present a protease cleavage site. Therefore, Ub/Ubls are the best spheres for the introduction of other protease cleavage sites for the design of protease substrates for anchoring and presentation to respective proteolytic enzymes. This property of easy access by protease of the protease substrates of the invention is believed to be responsible for higher affinity and increased signal to noise ratios of the enzymatic reactions.

The present invention is based in part upon the findings that a substrate of the present invention (a ubiquitin-luciferin conjugate) resulted in an unexpected increase in measured activity from a variety of different deubiquitinases (DUBs), as compared to Ub-AMC, Ub-rhodamine110, and Z-Arg-Leu-Arg-Gly-Gly-luciferin substrates (the sequence Arg-Leu-Arg-Gly-Gly is SEQ ID NO: 1). The attachment of luciferase substrates to ubiquitin and ubiquitin-like proteins results in highly sensitive substrates and assays for all DUBs, and, in particular, permits the high throughput screening (HTS) assay of certain DUBs for which no such assay was previously available. Previously, such DUBs were only assayable by the laborious and time-intensive analysis of polyubiquitin cleavage by western blotting. Depending upon the DUB, the ubiquitin-luciferin substrate of the present invention yielded a 10- to 1000-fold increase in signal-to-background ratios (S/B) over the fluorophore-conjugated substrates (Ub-AMC, Ub-rhodamine110) or the C-terminal Ub peptide-luciferin conjugate substrate (Z-RLRGG (SEQ ID NO: 1)-LUC). This surprising increase in sensitivity is likely to be partly due to an improved signal-to-background over fluorescent adducts and to differences in specificity for a tested isopeptidase between a Ub/Ubl protein substrate of the present invention and a short peptide substrate such as Z-RLRGG (SEQ ID NO: 1)-LUC.

In some embodiments, the protease substrates of the present invention comprise a Ub or Ubl protein moiety that is operably linked (e.g., by covalent linkage) by its C-terminus, to a luciferase substrate moiety (e.g., luciferin or coelenterazine). In some embodiments, a substrate comprising a Ub/Ubl-luciferase substrate conjugate serves as a reporter substrate for proteolytic enzymes, in particular its corresponding Ub/Ubl proteolytic enzymes. In those embodiments, the Ub/Ubl proteolytic enzymes cleave the linkage between the Ub/Ubl protein moiety and the luciferase substrate, rendering the luciferase substrate available for action by a luciferase to generate light.

In some embodiments, protease substrates of the present invention comprise a Ub or Ubl protein moiety that has been modified at its C-terminus to accommodate a particular protease cleavage recognition site (e.g., of a protease other than a DUB). In particular embodiments, the C-terminus of the Ub/Ubl moiety (e.g., the last five amino acids Arg-Leu-Arg-Gly-Gly (SEQ ID NO: 1)) is extended to a longer structure in order to accommodate the optimal sequence preferred by a protease other than a DUB. In some embodiments, a protease cleavage site of interest is added to the C-terminus of the Ub or Ubl, or the C-terminal peptide sequence of the Ub or Ubl (e.g., the last five amino acids Arg-Leu-Arg-Gly-Gly (SEQ ID NO: 1) or a C-terminal portion thereof) is replaced with a particular protease cleavage site. Thus, the modified C-terminus of the Ub or Ubl (having either a replacement or added protease cleavage site) may be operably linked, via the C-terminus, to the luciferase substrate moiety. In those embodiments, the specific protease for the specific protease cleavage site, cleaves the linkage between the Ub/Ubl moiety and the luciferase substrate, rendering the luciferase substrate available for action by a luciferase to generate light.

The protease substrates of the present invention provide improved sensitivity and utility over existing technology, in part based upon two distinct, yet related, properties of the luciferase/luciferase substrate reaction. First, luciferase enzyme specificity for its substrate is such that recognition and excitation of the luciferase substrate moiety, while still covalently linked to the Ub/Ubl moiety of the protease substrate, should be below the limits of detection. Secondly, excitation of the luciferase substrate (e.g., luciferin or coelenterazine), and subsequent light generation, can only be achieved enzymatically, not photometrically. Therefore, background light emission from the Ub/Ubl-luciferase substrate conjugate (the protease substrate) is non-existent.

As used herein, a “luciferase substrate” is any agent that can be acted upon by a luciferase, including, but not limited to a luciferin or a coelenterazine.

In particular embodiments of the methods of the present invention, a Ub/Ubl-amino-luciferase substrate conjugate serves as a reporter substrate for proteases, in particular its corresponding Ub/Ubl-specific protease. For example, cleavage of a Ub/Ubl-amino-luciferase substrate by an isopeptidase on the C-terminal end of the Ub/Ubl (e.g., after the C-terminal Gly-Gly sequence, the last two amino acids of the Ub/Ubl moiety of the conjugate), generates free amino-luciferase substrate. In other embodiments, proteases having other cleavage sites are assayed using Ub/Ubl-amino-luciferase substrate conjugates having a Ub or Ubl protein moiety that has been modified at its C-terminus to accommodate a particular protease cleavage recognition site that may be different in amino acid sequence and/or in length from the C-terminal peptide sequence of the Ub or Ubl (Arg-Leu-Arg-Gly-Gly (SEQ ID NO: 1)). In those embodiments, the protease cleaves the Ub/Ubl-amino-luciferase substrate after the C-terminal sequence of the particular protease's cleavage site to generate the free luciferase substrate.

In particular embodiments, the luciferase substrate is amino-luciferin. The terms “amino-luciferin” and “amino-luciferase substrate” may be used interchangeably herein. The amino-luciferase substrate can be acted upon by any luciferase, to generate light (luminescence) (de Wet et al., 1987, Mol. Cell. Bio., 7:725-737). In a particular embodiment, the Ub/Ubl moiety is conjugated at the C-terminus (e.g., after a Gly-Gly cleavage site) to luciferin, so that cleavage on the C-terminal end of the Ub/Ubl moiety by a protease liberates free amino-luciferin. In another embodiment, the Ub/Ubl moiety is conjugated at the C-terminus (e.g., after a Gly-Gly cleavage site) to coelenterazine, so that cleavage on the C-terminal end of the Ub/Ubl moiety by a protease, liberates free coelenterazine.

Luciferins are a class of light-emitting biological pigments that cause bioluminescence. Luciferins are found in many organisms, including firefly beetles, bacteria, and aquatic organisms, including snails, squid, fish, shrimp, dinoflagellates, jellyfish, sea pansies, and copepods. The luciferin found in members of the Lampyridae family of beetle insects is typically referred to as firefly luciferin. Coelenterazine is the luciferin molecule that is found in many bioluminescent aquatic organisms. Coelenterazine may serve as the substrate for the luciferases found in such aquatic organisms, including, but not limited to, Aequorea victoria, Renilla reniformis, and Gaussia princeps. Luciferin is widely available and can be prepared from the firefly as described in U.S. Pat. No. 4,826,989. Additional luciferin derivatives and their production are described in U.S. Pat. No. 5,035,999 and U.S. Pat. No. 5,098,828. Luciferin can be detected by any luciferase including without limitation, the firefly (Photinus pyralis) enzyme luciferase. Coelenterazine can be detected by any luciferase including, without limitation, those isolated from Aequorea victoria, Renilla reniformis and Gaussia princeps, or produced recombinantly from corresponding gene sequences derived from these organisms (see, e.g., U.S. Pat. No. 6,436,682). In addition to those listed above, any luciferase enzyme, including thermostable luciferases (see, e.g., U.S. Pat. No. 6,602,677 and U.S. Pat. No. 5,229,285), can be used in the methods of the present invention. Luciferase enzymes are available commercially and can be isolated or recombinantly produced using techniques well known to those of skill in the art.

Luciferase technology and methods of detecting luminescence are well known to those of skill in the art. Assays and techniques using luciferase and luciferase substrates are described in, for example, U.S. Pat. No. 5,238,179.

Methods of the present invention are amenable to high-throughput screening (HTS) formats, since the use of bioluminescent assays is a standard platform known in the art for HTS (see, e.g., Fan et al., 2007, Assay Drug Dev. Technol., 5:127-136).

In yet another embodiment of the instant invention, the substrates of the instant invention may be used to determine amino acid sequence recognized by a proteolytic enzyme. The Ub/Ubl moiety may be modified to include an amino acid sequence of interest (e.g., the sequence is added to the C-terminus of the Ub/Ubl or the Ub/Ubl cleavage site is modified/replaced to be the amino acid sequence of interest) and the ability of the protease to cleave the substrate is monitored as described herein, wherein luciferase activity corresponds to the ability of the protease to cleave the amino acid sequence of interest. In a particular embodiment, a panel of substrates is generated comprising more than one substrate having different cleavage sites (e.g., a peptide library of amino acid sequences (e.g., 1, 2, 3, 4, 5, 7, or 10 amino acids in length)) to screen for those cleaved by the proteolytic enzyme of interest.

The methods of the instant invention may be performed within a cell. DUB or protease activity may be measured in biological tissue or in cultured cells expressing a luciferase gene (e.g., transfected). Luciferase expressing cells (e.g., constitutively expressing cells) are known in the art. Such luciferase expressing tissue or cells can be used with assays of the present invention for the assay of DUB or protease activity. For example, at least one Ub/Ubl-amino-luciferase substrate conjugate or other protease substrate of the present invention may be delivered inside cells, optionally permeabilized. The free luciferase substrate, generated by the action of a DUB or protease is rapidly consumed by the endogenous luciferase expressed in the cell or tissue, and the cell or tissue emits light that is quantified. Alternatively, cell lysates can be incubated with at least one Ub/Ubl protease substrate or protease substrate of the invention and the protease activity is measured. These methods allow for the determination of the total pool and activities of all the DUBs in a cell that recognize the particular cleavage site(s).

### Protease Substrates

The protease substrates of the present invention comprise a first moiety (or portion) that comprises a ubiquitin protein (Ub) or a ubiquitin-like protein (Ubl) (also referred to herein as the “Ub/Ubl”), and a second moiety (or portion) that comprises a luciferase substrate. In a particular embodiment, the Ub or Ubl is covalently linked via an amide bond or linkage to the luciferase substrate. The C-terminus of the Ub/Ubl moiety comprises the protease cleavage site. In some embodiments, the cleavage site is the Ub/Ubl cleavage site (e.g, the five amino acid sequence Arg-Leu-Arg-Gly-Gly (SEQ ID NO: 1)). In some embodiments, the Ub/Ubl cleavage site is replaced with the cleavage site of another protease. In yet other embodiments, the first moiety comprises the Ub/Ubl plus the cleavage site of another protease added to the C-terminus of the Ub/Ubl. In such embodiments, the C-terminus of the Ub/Ubl moiety is altered in amino acid sequence and/or length from the five amino acid sequence Arg-Leu-Arg-Gly-Gly (SEQ ID NO: 1).

As used herein, the terms “cleavage recognition site” and “cleavage site” are used interchangeably with respect to the amino acid residues recognized and cleaved by a protease. In a particular embodiment, a cleavage site is about 1-10 amino acids, more particularly about 2 to about 5 amino acids. Any proteolytic enzyme can be assayed by the methods of the invention. The cleavage recognition site for a protease of interest is incorporated into the C-terminus of the Ub/Ubl moiety, such that the cleavage recognition site is covalently linked to the second moiety, i.e., the luciferase substrate. In some embodiments, e.g., in the case of certain Ub/Ubl proteolytic enzymes, the cleavage recognition site is the C-terminal five amino acids Arg-Leu-Arg-Gly-Gly (SEQ ID NO: 1) of the Ub/Ubl moiety. In other embodiments, the cleavage site of the protease of interest may be attached following the C-terminal amino acids of the Ub/Ubl moiety or may replace some portion (or all) of the C-terminal amino acids of the Ub/Ubl moiety. Standard genetic engineering techniques can be used to construct expression libraries for the random generation of Ub and Ubl proteins having different sequences of C-terminal linear peptide extensions. Modified Ub/Ubl proteins having optimal cleavage sites for particular proteases can be selected from the random library by standard techniques.

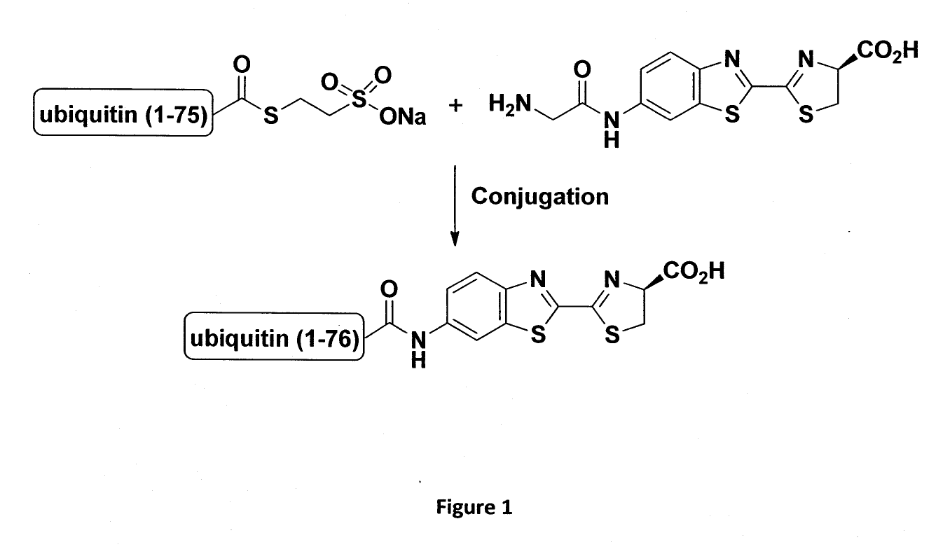

The instant invention also encompasses methods of synthesizing the protease substrate (see, e.g., FIG. 1). In a particular embodiment, the method comprises conjugating a thioester derivative of the Ub/UBL with a glycyl-amino-luciferase substrate. In a particular embodiment, the thioester derivative comprises mercaptoethanesulfonic acid. The methods of synthesis are superior (e.g., simpler, shorter, better yield) to chemical synthesis methods (see, e.g., Oualid et al. (2010) Angew. Chem. int. Ed., 49:10149:10153).

### Ubiquitin and Ubiquitin-Like Proteins

Ubiquitin (Ub) and ubiquitin-like (Ubl) proteins are well known in the art. In a particular embodiment, the full-length Ub or a Ubl is used in the conjugates of the instant invention. In another embodiment, a fragment of Ub or a Ubl is used in the conjugates of the instant invention. The Ub or Ubl fragments are active as substrates for their corresponding isopeptidase or corresponding protease, according to a particular incorporated C-terminal cleavage recognition site. Ub or Ubl fragments minimally include the C-terminus of the Ub or Ubl in order to provide the cleavage site to the isopeptidase. In a preferred embodiment, the Ub or Ubl fragment is greater than 5 amino acids in length. In particular embodiments, the Ub or Ubl fragment comprises 25%, 30%, 40%, 50%, 60%, 70%, 75%, 80%, 85%, 90%, 95%, 97%, 99% or more of the full-length Ub or Ubl, wherein the fragment comprises the C-terminus.

In a particular embodiment, the conjugate of the instant invention comprises a full-length (76 amino acids) ubiquitin protein. An exemplary amino acid sequence of ubiquitin is the mature human ubiquitin:
MQIFVKTLTGKTITLEVEPSDTIENVKAKIQDKEGIPPDQQRLIFAGKQLEDGR TLSDYNIQKESTLHLVLRLRGG (SEQ ID NO: 2),
   which is derived by post-translational processing of the naturally occurring human ubiquitin precursor, disclosed at GenBank Accession No CAA44911 (Lund et al., 1985, J. Biol. Chem., 260:7609-7613).

Ub and Ubls suitable for the methods and substrates of the present invention can come from any species including, without limitation, human and yeast. Any ubiquitin or Ubl can be used in the substrates and methods of the present invention for detecting activity of a cognate isopeptidase. Any ubiquitin or Ubl can be used in the substrates and methods of the present invention for detecting activity of other proteolytic enzymes, such as proteases having cleavage recognition sites that are different from the C-terminus of a Ub or Ubl. In embodiments for detecting such proteases having cleavage recognition sites that are different from the C-terminus of a Ub or Ubl, the C-terminus of the Ub or Ubl may be modified to comprise specific protease cleavage sites. In a particular embodiment, the Ub or Ubl of the conjugate is the mature form of the protein, i.e., the form of the protein after the precursor has been processed by a hydrolase or peptidase. In particular embodiments, the Ub/Ubl is a mammalian ubiquitin, more particularly, a human ubiquitin or Ubl. Ubls include, without limitation, small ubiquitin like-modifier-1 (SUMO), SUMO-2, SUMO-3, SUMO-4, ISG-15, HUB1 (homologous to ubiquitin 1; also known as UBL5 (ubiquitin-like 5)), APG12 (autophagy-defective 12), URM1 (ubiquitin-related modifier 1), NEDD8 (RUB1), FAT10 (also known as ubiquitin D), and APG8.

Amino acid sequences of Ubls and nucleic acid sequences encoding Ubls are known in the art. Amino acid and nucleotide sequences of SUMO proteins are provided, for example, in U.S. Pat. No. 7,060,461 and at GenBank Accession Nos. Q12306 (SMT3; amino acids 1-98 is the mature form), P63165 (SUMO1; precursor shown, mature form ends in GG), NM_{—}001005781.1 (SUMO1; precursor shown, mature form ends in GG), NP_{—}003343.1 (SUMO1; precursor shown, mature form ends in GG), NM_{—}006937.3 (SUMO2; precursor shown, mature form ends in GG), NM_{—}001005849.1 (SUMO2; precursor shown, mature form ends in GG), NM_{—}006936.2 (SUMO3; precursor shown, mature form ends in GG), and NM_{—}001002255.1 (SUMO4; precursor shown, mature form ends in GG). GenBank Accession No. CAI13493 provides an amino acid sequence for URM1. GenBank Accession No. NP_{—}001041706 provides an amino acid sequence for UBL5 (aka HUB1) (amino acids 1-72 represent the mature form). GenBank GeneID No. 4738 and GenBank Accession No. NP_{—}006147 provide amino acid and nucleotide sequences of NEDD8 (RUB1) (precursor shown, mature form ends in LRGG). GenBank Accession No. P38182 provides an amino acid sequence of yeast ATG8 (aka APG8) (precursor shown, mature form ends in FG). GenBank Accession Nos. BAA36493 and P38316 provide amino acid sequences of human and yeast ATG12 (aka APG12), respectively (human precursor shown, mature form ends in FG). GenBank Accession Nos. AAH09507 and P05161 provide amino acid sequences of human and yeast ISG15 ubiquitin-like modifier, respectively (precursors shown, mature form ends in GG). GenBank Accession No. AAD52982 provides an amino acid sequence of ubiquitin D (aka human FAT10, UBD-3, UBD, GABBR1).

Modifications to the C-terminal end of the Ub/Ubl moiety to introduce cleavage recognition sites for proteases of interest can be carried out by methods well known to the art, including but not limited to recombinant methods utilizing E. coli. Libraries of C-terminally modified Ub/Ubl moieties comprising cleavage recognition sites of proteases can be generated by a variety of recombinant methods well known to the art. Structurally diverse libraries of such C-terminally modified Ub/Ubl moieties comprising cleavage recognition sites of a wide variety of proteases can be created by methods well known to the art.

Recombinant engineering and expression methods are well known to the art and are described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.

Cleavage recognition sites for proteases are well known to the art. Any known protease cleavage recognition site can be attached or added to the C-terminal end of a Ub/Ubl moiety to create a substrate for a cognate protease. In some embodiments, the cleavage recognition site replaces the C-terminal amino acids of the Ub/Ubl moiety, and in some embodiments, the cleavage recognition site is added C-terminally to the C-terminus of the Ub/Ubl moiety. In a particular embodiment, the proteolytic enzyme cleaves C-terminal to its recognition sequence.

In some embodiments, a caspase cleavage recognition site is attached or added to the C-terminal end of a Ub/Ubl moiety to create a substrate for a caspase protease. Caspases cleave a substrate comprising aspartate at the C-terminal side of the aspartate. In embodiments where a caspase cleavage recognition site is attached or added to the C-terminal end of a Ub/Ubl moiety to create a substrate for a caspase protease, an aspartate may be the C-terminal amino acid of the Ub/Ubl moiety, and this aspartate may be covalently linked to the luciferase substrate moiety.

Non-limiting examples of caspase cleavage recognition sites useful in the materials and methods of the invention include the following: DEVD (SEQ ID NO:3) recognition site for caspase 3 and 7, YVAD (SEQ ID NO:4) recognition site for caspase 1, WEHD (SEQ ID NO:5) recognition site for caspase 1, 4 and 5, VDVAD (SEQ ID NO:6) recognition site for caspase 2, LEHD (SEQ ID NO:7) recognition site for caspase 4, 5, 9 and 11, VEID (SEQ ID NO:8) recognition site for caspase 6, VEVD (SEQ ID NO:9) recognition site for caspase 6, VEHD (SEQ ID NO:10) recognition site for caspase, IETD (SEQ ID NO:11) recognition site for caspase 8, AEVD (SEQ ID NO:12) recognition site for caspase 10, LEXD (SEQ ID NO:13) recognition site for caspase 8 and 10, where X is any amino acid, VEXD (SEQ ID NO:14) recognition site for caspase 8, where X is any amino acid, IEHD (SEQ ID NO:15) recognition site for caspase 11, PEHD (SEQ ID NO:16) recognition site for caspase 11, (I/L/V/P)EHD (SEQ ID NO:17) recognition site for caspase 11, DEHD (SEQ ID NO:18), LETD (SEQ ID NO:19) recognition site for caspase, and (L/V)EXD (SEQ ID NO:20) recognition site for caspase 8, where X is any amino acid. A non-limiting example of a consensus cleavage recognition site for a caspase is X₁-X₂-X₃-D (SEQ ID NO: 21), wherein X₁ is Y, D, L, V, I, A, W, or P; X₂ is V or E; and X₃ is any amino acid.

In some embodiments, a trypsin cleavage recognition site is attached or added to the C-terminal end of a Ub/Ubl moiety to create a substrate for a trypsin protease. Trypsins cleave peptide chains mainly at the carboxyl side of the amino acids lysine or arginine. In embodiments where a trypsin cleavage recognition site is attached or added to the C-terminal end of a Ub/Ubl moiety to create a substrate for a trypsin protease, a lysine or arginine may be the C-terminal amino acid of the Ub/Ubl moiety, and this lysine or arginine may be covalently linked to the luciferase substrate moiety.

In some embodiments, a tryptase cleavage recognition site is attached or added to the C-terminal end of a Ub/Ubl moiety to create a substrate for a trypsin protease. Tryptases cleave peptide chains mainly at the carboxyl side of the amino acids lysine or arginine. In embodiments where a tryptase cleavage recognition site is attached or added to the C-terminal end of a Ub/Ubl moiety to create a substrate for a tryptase protease, a lysine or arginine may be the C-terminal amino acid of the Ub/Ubl moiety, and this lysine or arginine may be covalently linked to the luciferase substrate moiety.

In some embodiments, a chymotrypsin cleavage recognition site is attached or added to the C-terminal end of a Ub/Ubl moiety to create a substrate for a chymotrypsin protease. Chymotrypsins cleave peptide chains mainly at the carboxyl side of the amino acids tyrosine, phenylalanine or tryptophan. In embodiments where a chymotrypsin cleavage recognition site is attached or added to the C-terminal end of a Ub/Ubl moiety to create a substrate for a chymotrypsin protease, a tyrosine, phenylalanine, or tryptophan may be the C-terminal amino acid of the Ub/Ubl moiety, and this tyrosine, phenylalanine, or tryptophan residue may be covalently linked to the luciferase substrate moiety.

In some embodiments, a cathepsin cleavage recognition site is attached or added to the C-terminal end of a Ub/Ubl moiety to create a substrate for a trypsin protease. Cathepsin cleavage recognition sites are well known to the art. In embodiments where a cathepsin cleavage recognition site is attached or added to the C-terminal end of a Ub/Ubl moiety to create a substrate for a cathepsin protease, the amino acid residue after which a cathepsin cleaves may be the C-terminal amino acid of the Ub/Ubl moiety, and that amino acid may be covalently linked to the luciferase substrate moiety. Non-limiting examples of cleavage recognition sites for cathepsins, include consensus sited for cathepsin-D: Xaa-Xaa-Xaa-Xaa-hydrophobic-hydrophobic (SEQ ID NO: 22) and Xaa-Xaa-Xaa-Xaa-Glu-hydrophobic (SEQ ID NO: 23), where Xaa=any amino acid residue and hydrophobic=Ala, Val, Leu, Ile, Phe, Trp, Tyr; and consensus sites for cathepsin-L: Xaa-Xaa-Xaa-hydrophobic-Phe-Arg (SEQ ID NO: 24), Xaa-Xaa-Xaa-aromatic-Phe-Arg (SEQ ID NO: 25), Xaa-Xaa-Xaa-hydrophobic-Arg-Arg (SEQ ID NO:26), and Xaa-Xaa-Xaa-aromatic-Arg-Arg (SEQ ID NO: 27), where Xaa=any amino acid residue, hydrophobic=Ala, Val, Leu, Ile, Phe, Trp, Tyr, and aromatic=Phe, Trp, His, Tyr.

In some embodiments, a beta-secretase cleavage recognition site is attached or added to the C-terminal end of a Ub/Ubl moiety to create a substrate for a beta-secretase protease. In embodiments where a beta-secretase cleavage recognition site is attached or added to the C-terminal end of a Ub/Ubl moiety to create a substrate for a beta-secretase protease, the amino acid residue after which a beta-secretase cleaves may be the C-terminal amino acid of the Ub/Ubl moiety, and that amino acid may be covalently linked to the luciferase substrate moiety. In a particular embodiment, the beta-secretase cleavage site is VNL-DA (SEQ ID NO: 28)

In some embodiments, the Ub or Ubl is 100% identical in amino acid sequence to a wild-type Ub or Ubl. In other embodiments, the Ub or Ubl is a naturally or artificially-created variant sequence Ub or Ubl. In particular embodiments, variants of a particular Ub or Ubl have an overall amino acid sequence that is at least 75%, at least 80%, at least 85%, at least 90%, at least 95%, or at least 99% identical to the wild-type Ub or Ubl sequence. Those of skill in the art are aware of methods for determining sequence identity. Calculation of sequence identity can, for example, be performed by published algorithms. Alignment of sequences for comparison may be conducted by the local homology algorithm of Smith & Waterman, 1981, Adv. Appl. Math., 2:482, by the homology alignment algorithm of Needleman & Wunsch, 1970, J. Mol. Biol., 48:443, by the search for similarity method of Pearson & Lipman, 1988, Proc. Natl. Acad. Sci. U.S.A., 85:2444, by computerized implementations of these algorithms (GAP, BESTFIT, FASTA, and TFASTA in the Wisconsin Genetics Software Package, Genetics Computer Group, 575 Science Dr., Madison, Wis.), or by inspection.

Any variant Ub or Ubl protein is contemplated for use in the substrates and methods of the invention, with the proviso that the variant remains active for cleavage by a protease. Variant Ub and Ubl proteins include those having one or more amino acid substitutions, deletions or insertions in comparison to the wild-type Ub or Ubl. In particular embodiments, a Ub or Ubl protein may have one or more amino acid substitutions, deletions or insertions that can be conservative or non-conservative. As used herein, a “conservative” amino acid substitution/mutation refers to substituting a particular amino acid with an amino acid having a side chain of similar nature (i.e., replacing one amino acid with another amino acid belonging to the same group). A “non-conservative” amino acid substitution/mutation refers to replacing a particular amino acid with another amino acid having a side chain of different nature (i.e., replacing one amino acid with another amino acid belonging to a different group). Groups of amino acids having a side chain of similar nature are known in the art and include, without limitation, basic amino acids (e.g., lysine, arginine, histidine); acidic amino acids (e.g., aspartic acid, glutamic acid); neutral amino acids (e.g., glycine, asparagine, glutamine, serine, threonine, tyrosine, cysteine, alanine, valine, leucine, isoleucine, proline, phenylalanine, methionine, tryptophan); amino acids having a polar side chain (e.g., glycine, asparagine, glutamine, serine, threonine, tyrosine, cysteine); amino acids having a non-polar side chain (e.g., alanine, valine, leucine, isoleucine, proline, phenylalanine, methionine, tryptophan); amino acids having an aromatic side chain (e.g., phenylalanine, tryptophan, histidine); amino acids having a side chain containing a hydroxyl group (e.g., serine, threonine, tyrosine), and the like.

As used herein, “poly-ubiquitin” or “poly-ubiquitin-like” refers to a chain of ubiquitin or ubiquitin-like molecules comprising more than one ubiquitin or ubiquitin-like protein. As used herein, “mono-ubiquitin” or “mono-ubiquitin-like” refers to a single ubiquitin or ubiquitin-like protein. In the methods of the present invention, a mono- or poly-ubiquitin or a mono- or poly-ubiquitin-like protein can serve as the Ub/Ubl moiety of the protease substrate.

In some embodiments, the Ub/Ubl moiety is a poly-Ub or poly-Ubl molecule. Thus, poly-ubiquitin chains of, for example, 2, 3, 4 or more ubiquitin or Ubl molecules can serve as the Ub/Ubl moiety of the isopeptidase substrate. These polyubiquitin chains are formed through (isopeptide) linkages between the C-terminus of one Ub/Ubl and a specific lysine of the next Ub/Ubl. The lysine may be a lysine located at amino acid positions: 6, 11, 27, 29, 33, 48, or 63. In a particular embodiment, the linkages between Ub or Ubl molecules is not the natural Gly-Gly linkage. For example, the linkage can be generated with MESNa chemistry (not enzymatically) in order to have the linkage sequence be, for example, Ala-Ala, and not the natural Gly-Gly. The last Ub/Ubl in the chain (having the C-terminus linked to the luciferase substrate) would be the only Ub/Ubl that has Gly-Gly. This would essentially create a substrate that has potentially better binding properties over mono-Ub/mono-Ubl, but still can be cleaved at the end to release the luciferase substrate.

In some embodiments, more than one protease substrate may be used for protease activity detection. In particular embodiments, more than one peptidase substrate may be used for peptidase activity detection. In particular embodiments, more than one caspase or tryptase or trypsin substrate may be used for caspase or tryptase or trypsin activity detection.

### Preparation of Protease Substrates

Substrates of the present invention can be produced, for example, by a modification of a technique for coupling fluorophores to the C-terminus of ubiquitin (Hassiepen et al., 2007, Analyt. Biochem., 371:201-201). In particular embodiments, the Ub or Ubl protein is recombinantly produced as a Ub/Ubl-intein fusion protein with a C-terminal affinity tag (such as hexahistidine) for purification (Wilkinson et al., 2005, Meth. Enzymol., 399:37-51). In a particular embodiment, when using this intein method to recombinantly produce a fusion protein of a Ub or Ubl protein with intein and an affinity at the C-terminus, the nucleic acid encoding the Ub/Ubl-intein-tag does not encode the final C-terminal glycine residue of the Ub/Ubl protein. The intein moiety of the resultant recombinant protein self-splices such that the fusion protein commits a N—S acyl rearrangement at the C-terminal residue of the Ub or Ubl and the N-terminal cysteine of the intein portion, resulting in a thioester. The Ub/Ubl intein fusion protein can then be immobilized on affinity resin and cleaved with mercatoethanesulfonic acid (MESNA), to generate a thioester derivative (Ub/Ubl-MESNa).

Glycyl-D-amino-luciferin can be synthesized and purified using a slightly modified form of published protocols (Shinde et al., 2006, Biochem., 45:11103-11112; White et al. 1966, J. Am. Chem. Soc., 88:2015-2019). Purified Ub/Ubl-MESNa can be conjugated to glycyl-D-amino-luciferin using a modified version of published protocols (Wilkinson et al., 2005, supra). Specifically, the purified Ub/Ubl-MESNa and the glycyl-D-amino-luciferin are mixed in a solvent and the conjugation reaction is carried out for a shorter period of time at a higher temperature. These three alterations yield optimal solubility of the luciferin molecule and optimal efficiency of the conjugation reaction. Thus, a Ub/Ubl-amino-luciferin substrate can be produced using the following conditions: a solvent of 2:1 DMF/DMSO, plus 2% triethylamine, a temperature of 40° C., and a duration of conjugation reaction of 16 hours.

The methods and substrates of the present invention provide greater sensitivity, higher specificity, enhanced catalytic efficiency, and higher S/B values, allowing for increased lower limits of detection of proteases, over existing protease activity detection methodologies. Specifically, we have found that the Ub-amino-luciferin substrate yields about 1000-fold greater S/B compared to Z-RLRGG (SEQ ID NO: 1)-amino-luciferin substrate for UCHL3 activity. The Ub-amino-luciferin substrate yields a range of about 10- to about 1000-fold greater S/B compared to Z-RLRGG (SEQ ID NO: 1)-amino-luciferin for activity of a panel of 9 isopeptidases (Otub2, JosD1, JosD2, AMSH, Ataxin3, Ataxin3-like, UCHL5, USP20, and USP14). The Ub-amino-luciferin substrate yields about 60- to about 120-fold greater S/B compared to Ub-AMC for UCHL3, USP7, USP8, and USP2core activities. The Ub-amino-luciferin substrate yields more than 30-fold greater S/B compared to Ub-rhodamine110 for UCHL3, USP7, USP8, and USP2core activities. The Ub-amino-luciferin substrate yields about 50-fold greater S/B compared to Ub-AMC for UCHL3 activity. The Ub-amino-luciferin substrate yields about 200-fold greater S/B compared to Ub-rhodamine110 for UCHL3 activity.

In some embodiments, the protease substrates of the present invention when used in the methods of the present invention may yield luminescence having a signal to background ratio that is about 10- to about 1000-fold greater than the S/B for a corresponding assay wherein the first moiety in the isopeptidase substrate is a C-terminal peptide of Ub or a Ubl or the second moiety is a fluorophore, such as AMC or rhodamine110.

In some embodiments, the protease substrates of the present invention yield luminescence having a signal to background ratio that is at least about 10-fold greater, at least about 20-fold greater, at least about 30-fold greater, at least about 40-fold greater, at least about 50-fold greater, at least about 60-fold greater, at least about 70-fold greater, at least about 80-fold greater, at least about 90-fold greater, at least about 100-fold greater, at least about 125-fold greater, at least about 150-fold greater, at least about 200-fold greater, at least about 300-fold greater, at least about 400-fold greater, at least about 500-fold greater, least about 600-fold greater, least about 700-fold greater, least about 800-fold greater, least about 900-fold greater, or least about 1000-fold greater than the S/B for a corresponding assay wherein the first moiety in the isopeptidase substrate is a C-terminal peptide of Ub or a Ubl (e.g., the C-terminal petapeptide) and/or the second moiety is a fluorophore, such as AMC or rhodamine110.

As use herein, the phrase “C-terminal peptide of a Ub or a Ubl” refers to a short peptide that is at least 5 amino acids in length and that is the C-terminus of a ubiquitin or a ubiquitin-like protein. Specifically this short peptide comprises the C-terminal residues of the mature form of a ubiquitin or ubiquitin-like protein. An exemplary “C-terminal peptide of Ub or a Ubl” is RLRGG (Arg-Leu-Arg-Gly-Gly; SEQ ID NO: 1).

Methods and substrates of the present invention can be used to detect the activity or presence of a wide variety of proteases from any organism (e.g., animals, plants, yeast, viruses, bacteria), including but not limited to isopeptidases, including deubiquitinating enzymes or a ubiquitin-like protein (Ubl)-specific proteases (Ulp); serine proteases, such as trypsins, chymotrypsins, and tryptases; threonine proteases, such as proteasome catalytic subunits; cysteine proteases, such as caspases and cathepsins; aspartate proteases, such as cathepsins, pepsins, renins, and beta-secretase (BACE); metalloproteases, such as members of the family ADAMTS (A Disintegrin And Metalloproteinase with Thrombospondin Motifs) and A Disintegrin and Metalloprotease Domain (ADAM); and glutamic acid proteases, such as scytalidoglutamic peptidase.

In some embodiments, methods and substrates of the invention are used to detect the activity or presence of a caspase, cathepsin, chymotrypsin, beta-secretase, trypsin, tryptase, serine protease, pro-hormone precursor, subtilisin/kexin-like pro-hormone convertase, carboxypeptidase, A Disintegrin-like And Metalloprotease domain (reprolysin-type) with ThromboSpondin type I motif (ADAMTS), A Disintegrin and Metalloprotease Domain (ADAM), cystein aspartase, aspartic proteinase, Matrix Metalloproteinase (MMP), RNA-dependent RNA polymerase, N-terminal nucleophile (Ntn) hydrolase, 4-oxalocrotonate tautomerase, chorismate synthase, β-lactam acylase, reverse transcriptase, phospholipase, transcription factor, a viral reverse transcriptase, sigma transcription factor, Glutamine phosphoribosylpyrophosphate (PRPP) amidotransferase (GPATase), coagulation factor VIIa, coagulation factor Xa, coagulation IXa, coagulation XIa, Thrombin Acitivated Fibrinolysis Inhibitor a (TAFIa), plasmin, tissue plasminogen activator, 3Dpol RNA-dependent RNA polymerase, glutamine 5-phosphoribosyl-1-pyrophosphate amidotransferase, penicillin acylase, reverse transcriptase, chorismate synthase, tryptase, chymase, enterokinase, transcription factor OK, thrombin, dipeptidyl peptidase, HtrA2, neurophysin, vasopressin, furin, subtilisin-kexin-isozyme-1 (SKI-1), proprotein convertase subtilisin kexin 9 (PCSK9), carboxypeptidase B, carboxypeptidase Y, vWF-cleaving protease/ADAMTS 13, ADAM 1, ADAM 2, caspase, pepsin, rennin, cathepsin D, Mason-Pfizer monkey virus proteinase, MMP20, MMP26, glycosylasparginase, 20S proteasome β subunit, glutamine PRPP amidotransferase, YdcE, YwhB, cephalosporin acylase, CaMV reverse transcriptase, and phospholipase A₂.

In particular embodiments, the methods and substrates of the present invention are used to detect the activity or presence of a protease selected from a caspase, a trypsin, a tryptase, a cathepsin, a chymotrypsin, and a beta-secretase.

Methods and substrates of the present invention can be used to detect the activity or presence of a wide variety of isopeptidases from any organism, including but not limited to, the following deubiquitinating enzymes or a ubiquitin-like protein (Ubl)-specific proteases (Ulp): ULP1, ULP2, SENP1, SENP2, SENP3, SENP5, SENP6 (aka SUSP1, SSP1), SENP7, NEDD8-specific protease 1 (aka DEN1, Nedp1, Prsc2, SENP8), yeast YUH1, mammalian UCH-L1 (aka Park 5), UCH-L3, UCH-L5 (aka UCH37), USP1 (aka UBP), USP2 (aka UBP41), USP2core, USP2a, USP2b, USP3, USP4 (aka UNP, UNPH), USP5 (aka isopeptidase T, ISOT), USP6 (aka TRE2, HRP-1), USP7 (aka HAUSP), USP8 (aka UBPY), USP9, USP9Y (aka DFFRY), USP9X (aka DFFRX), USP10 (aka UBPO, KIAA0190), USP11 (aka UHX1), USP12 (aka USP12L1, UBH1), USP13 (aka ISOT3), USP14 (aka TGT), USP15, USP16 (aka UBP-M), USP18 (aka UBP43, ISG43), USP19 (aka ZMYND9), USP20 (aka VDU1, LSFR3A), USP21, USP22 (aka KIAA1063), USP23, USP24, USP25, USP26, USP27, USP28, USP29, USP30, USP32, USP33 (aka VDU2), USP34, USP35, USP36, USP37, USP38, USP40, USP42, USP44, USP46, USP49, USP51, JosD1 (aka KIAA0063), JosD2 (aka RGD1307305), AMSH, AMSHcore, Ataxin3 (aka ATX3, MJD, MJD1, SCA3, ATXN3), Ataxin3-like, Bap1 (UCHL2 or HUCEP-13), DUB-1, DUB-2, DUB1, DUB2, DUB3, DUB4, CYLD, CYLD1, FAFX, FAFY, OTUB1 (aka OTB1, OTU1, HSPC263), OTUB2 (aka OTB2, OTU2, C14orf137), OUT domain containing 7B (aka OTUD7B, Cezanne), KIAA0797, KIAA1707, KIAA0849, KIAA1850, KIAA1850, KIAA0529, KIAA1891, KIAA0055, KIAA1057, KIAA1097, KIAA1372, KIAA1594, KIAA0891, KIAA1453, KIAA1003, UBP1, UBP2, UBP3, UBP4, UBP5, UBP6, UBP7, UBP8, UBP41, UBP43, VCIP135, Tnfaip3 (aka A20), PSMD14 (aka POH1), COP9 complex homolog subunit 5 (aka CSN5, COPS5, JAB1) YPEL2 (aka FKSG4, and SARS CoV PLpro. Isopeptidases and their nucleic acid coding sequences are well known to those of skill in the art. For use in certain embodiments, isopeptidases can be isolated or recombinantly produced by methods well known in the art.

In particular embodiments, the methods and substrates of the present invention are used to detect the activity or presence of an isopeptidase selected from UCHL3, USP2core, USP7, USP8, USP34, Otub2, JosD1, JosD2, AMSH, Ataxin3, Ataxin3-like, UCHL5, USP20, USP14, ULP1, Ulp2, SENP1, SENP2, A20 and SENP5.

In some embodiments, methods of the present invention are practiced using a sample in which the activity of a protease may be detected. Samples may be from a variety of sources including animal or plant cell or cellular lysates, in vitro reaction mixtures, such as for drug screening purposes, including solutions and/or mixtures containing recombinantly produced isopeptidase, and bodily fluids or tissue samples taken from an animal such as a human.

Methods for Screening for Agents that Modulate Protease Activity

The present invention also provides methods for screening for agents that can modulate the activity of a particular protease of interest. In these methods, an appropriate protease substrate of the invention may be contacted with its corresponding protease in the presence and in the absence of one or more test agents. This contacting may take place under appropriate conditions for the protease to cleave the protease substrate and liberate the luciferase substrate, which will then generate a luminescent signal that can be detected and quantified. Any alteration or difference in the level of luminescence detected in the presence of the one or more test agent, as compared with the level of luminescence detected in the absence of the one or more test agent, will be an indication that the one or more test agent is capable of modulating the activity of the protease.

As used herein, “modulate” and “capable of modulating”, in reference to a test agent or agent, includes agents that can increase/enhance or inhibit/decrease/diminish the activity of a particular protease. Therefore, screening methods of the present invention are useful for identifying agents that can increase/enhance or inhibit/decrease/diminish the activity of a particular protease.

Any kind of compound or molecule may be tested as a candidate protease modulating agent in the methods of the present invention, including, but not limited to, natural or synthetic chemical compounds (such as small molecule compounds (including combinatorial chemistry libraries of such compounds)), extracts (such as plant-, fungal-, prokaryotic- or animal-based extracts), fermentation broths, organic and inorganic compounds and molecules, and biological macromolecules (such as saccharide-, lipid-, peptide-, polypeptide- and nucleic acid-based compounds and molecules). The activity of a modulator may be known, unknown, or partially known.

Candidate protease modulating agents may be evaluated for potential activity as inhibitors or enhancers (directly or indirectly) of a biological process or processes associated with a particular protease, such as for example, a particular Ub- or Ubl-specific isopeptidase (e.g., agonist, partial antagonist, partial agonist, inverse agonist, antagonist, antineoplastic agents, cytotoxic agents, inhibitors of neoplastic transformation or cell proliferation, cell proliferation-promoting agents, and the like) by inclusion in screening methods and assays described herein.

Agents identified as capable of modulating the activity of particular proteases using the methods of the present invention may useful for the preparation of drugs for the treatment of diseases or conditions associated with a particular protease, such as a Ub- or Ubl-specific isopeptidase or its corresponding Ub or Ubl, as well as for further dissecting the mechanisms of action of these enzymes.

Method for Diagnosing Diseases Associated with Proteases

The present invention also provides methods for diagnosing a disease or condition associated with a particular protease, such as, for example, a particular Ub- or Ubl-specific isopeptidase, where a sample from a subject suspected of having the disease or condition is contacted with an appropriate protease substrate of the present invention and with a luciferase, followed by detection of luminescence. The level of luminescence is indicative of the protease activity in the sample. The amount of protease activity in the sample can be compared to the amount of protease activity in a corresponding sample from a healthy control, wherein a modulation (e.g., increase or decrease) in the protease activity in the sample compared to healthy controls is indicative of the presence of a disease or disorder.

Many diseases or conditions are known to be associated with a protease, such as for example, a Ub- or Ubl-specific proteolytic enzyme. For example, diseases or conditions associated with a protease, such as for example, a Ub- or Ubl-specific proteolytic enzyme may be associated with altered enzyme levels, amounts, sequences and/or activities. Particular diseases or conditions associated with a protease, such as for example, a Ub- or Ubl-specific proteolytic enzyme include, but are not limited to, auto-immune, neoplastic, metabolic, vascular, neurodegenerative and other genetic diseases or conditions.

Methods of the present invention can be used to diagnose diseases or conditions in subjects of any organism, plant or animal, suspected of having a disease or condition associated with a particular protease, such as for example, a particular Ub- or Ubl-specific proteolytic enzyme. Therefore, the sample from the subject may include a cell or cells, a piece of tissue, cellular or tissue extract, or bodily fluid from such organism. In particular embodiments, the sample is from a human patient suspected of having a disease or condition associated with a particular protease, such as for example, a particular Ub- or Ubl-specific proteolytic enzyme.

Any disease or condition known to be associated with a protease, such as for example, a Ub- or Ubl-specific proteolytic enzyme can be detected using the methods of the present invention. Specific examples of the diseases of conditions associated with a Ub- or Ubl-specific proteolytic enzyme are cancer, e.g., breast, prostate, and cancers associated with von Hippel-Lindau disease which predisposes to a number of cancers such as hemangioblastomas, pheochromocytomas, and cystadenomas, as well as other diseases such as lupus, diabetes, IBD, Parkinson's disease and cardiovascular disease. Examples of proteolytic enzyme/isopeptidase/deubiquitinating enzymes associated with disease include the following.

### VDU1/2 and Cancer

von Hippel-Lindau disease is an hereditary cancer syndrome caused by germline mutations of the VHL gene (Sims, 2001, Curr. Opin. Neurol., 14:695-703). von Hippel-Lindau predisposes those with the disease to various tumors, including hemangioblastomas in the CNS and retina, clear cell renal carcinomas, pheochromocytomas of adrenals, pancreatic tumors, cystadenomas of the epididymis, and tumors of the inner ear (Li et al., 2002, J. Biol. Chem. 277:4656-62; Maher, et al., 1997, Medicine (Baltimore), 76:381-91). VHL protein (pVHL) associates with elongin C, elongin B, and cullin-2 to form a complex, VCB-CUL2, which acts as an ubiquitin E3 ligase (Lisztwan et al., 1999, Genes Dev., 13:1822-1833). Because mutated pVHL is associated with malignancies, the ligase can be considered to be a tumor suppressor and its substrates potential oncogenic molecules. Hypoxia-inducible factor (HIF-α), known to be a substrate of VCB-CUL2, plays a role in development of hemangioblastomas, and likely in tumor angiogenesis in general, via VEGF induction (Ohh et al., 2000, Nat. Cell Biol., 2(7):423-427; Tyers et al., 1999, Proc. Natl. Acad. Sci. USA, 96(22):12230-12232; Benjamin et al., 1997, Proc. Natl. Acad. Sci. USA, 94(16):8761-8766). Also among its substrates, is ubiquitin isopeptidase USP20 (aka VDU1), found by yeast 2-hybrid screening to interact with pVHL. A highly homologous protease, USP33 (aka VDU2), is also known; although it has not been studied in terms of pVHL association, VDU2 has physiological substrates in common with VDU1 (Curcio-Morelli et al., 2003, J. Clin. Invest., 112(2):189-196). The β-domain region of pVHL, a site of naturally occurring mutations, is the locus of VDU1 interaction, and VDU1 may be co-immunoprecipitated in the VCB-CUL2 complex. The ubiquitination and degradation of VDU1 by a pVHL-dependent pathway is abrogated by VHL mutations that disrupt interactions with VDU1. Thus, targeted degradation of VDU1 by pVHL is important in suppressing tumor formation and/or maintenance, and VDU1 may have oncogenic activity that is uncovered in the absence of the functional ligase. VDU1, therefore, is important in neoplastic disease characterized by mutated pVHL (100% of patients with VHL (autosomal dominant) disease), and 50-80% of the far larger number of patients with sporadic renal clear cell carcinoma (Stolle et al., 1998, Hum. Mutat., 12(6):417-423; Gnarra et al., 1994, Nat. Genet., 7(1):85-90.). Inhibition of VDU1 functionally mimics the activity of the wild type tumor suppressor pVHL.

USP7 (also Known as HAUSP) and USP2a and Cancer

Deubiquitinating enzymes may serve to spare certain proteins, or at least prolong their cellular lifetime by removing the initial ubiquitin tag, thereby preventing proteasomal degradation. USP7 is known to stabilize the tumor suppressor p53 (Li et al., 2002, supra). USP2a has been implicated in the regulation of fatty acid synthase (FAS), a molecular signature of prostate cancer (Rossi et al., 2003, Mol. Cancer Res., 1(10):707-715; Agostini et al., 2004, Oral. Oncol., 40(7):728-735; Graner et al., 2004, Cancer Cell, 5(3):253-61.). USP2a is androgen-regulated and over-expressed in prostate cancer, and is thus an oncogenic protein. Thus, depending on the roles of their substrates, deubiquitinating enzymes can be either activated or inhibited to achieve therapeutic effect.

### Isopeptidase T and Cardiovascular Disease

The deubiquitinating enzyme Isopeptidase T is down-regulated in patients with chromosome 22q 11 deletion syndrome, which encompasses a variety of heart defects (Yamagishi et al., 1999, Science, 283(5405):1158-1161). Along with UFD1, isopeptidase T is down-regulated in myocytes from patients with heart failure (see, e.g., Kostin et al., 2003, Circ. Res., 92(7):715-724). This isopeptidase is known to remove polyubiquitin chains from ubiquitin-protein conjugates and stimulate protein degradation, and its absence results in accumulation of polyubiquitinated proteins and a disruption of the ubiquitin-proteasome degradation pathway, thereby leading to autophagic cell death (Hadari et al., 1992, J. Biol. Chem., 267(2):719-727; Johnson et al., 1995, Biol. Chem., 270(29):17442-17456; Stefanis et al., 2001, J. Neurosci., 21(24):9549-9560).

### JAMM Motif Isopeptidase AMSH and Pulmonary Disease and Cancer

A JAMM domain-containing protein is linked with the signal transduction associated with endosomal sorting, i.e., trafficking between the membrane and endosomalaysosomal compartments, of the EGF receptor (EGFR). This protein, AMSH (Associated Molecule with the SH3-domain of STAM), is a protein that regulates receptor sorting at the endosome (McCullough et al., 2004, J. Cell Biol., 166(4):487-492; Clague et al., 2001, J. Cell Sci., 114(Pt 17):3075-3081). The EGFR regulates numerous cellular functions by initiating signal transduction cascades (Lockhart et al., 2005, Semin. Oncol., 32(1):52-60; von Ahsen et al., 2005, Chembiochem, 6(3):481-490; Leroy et al., 1998, Nature, 395(6701):451-452; Spano et al., 2005, Ann. Oncol., 16(2):189-194.). During the cellular lifetime of the EGFR, it recycles from membrane to early (sorting) endosome, before finally being selected for sorting to the late endosome and lysosome, where it is degraded by acid proteases. The EGFR participates in signal transduction both at the membrane and in the early endosome compartment. While much of the signaling is concerned with regulation of cell growth and other functions, one component of signal transduction regulates trafficking of the EGFR itself The E3 ligase Cbl mediates ubiquitination of phosphorylated EGFR. Subsequent signaling events result in degradation of the receptor in late endosomes/lysosomes. Ub-EGFR is recognized by the protein Hrs at the endosomal surface, and further interactions with the endosomal-associated complex required for transport (ESCRT) mediated by ubiquitin result in translocation to internal vesicles of the multi-vesicular body (MVB), committing EFGR to protease degradation in the lysosome. Degradation, the end result of Cbl mediated ubiquitination of EGFR, may be abrogated by a ubiquitin isopeptidase, AMSH, e.g., ablation of AMSH activity by incubation of cells with siRNA leads to increased EGFR degradation; purified AMSH de-ubiquitinates EGFR-Ub in vitro (McCullough et al., 2004, supra). GFR kinase inhibitors and receptor binding antagonists are currently in clinical trial for various cancers (Ciardiello et al., 2001, Clin. Cancer Res., 7(10):2958-2970; LoRusso et al., 2003, Clin. Cancer Res., 9(6):2040-2048). Other disease areas with critical unmet needs are also associated with EGFR activity, such as airway inflammation and mucous hypersecretion associated with bronchial asthma. While asthma is a multifactorial disease, damage of the bronchial epithelium associated with leukocyte infiltration and increased airway responsiveness are consistent features (Puddicombe et al., 2000, FASEB J., 14(10):1362-1374.). The EFGR system has been postulated to play important roles in the growth and differentiation of epithelial and connective tissue cell types in the lung. The EGFR and its ligands are elevated during the pathogenesis of asthma, and induction of this system correlates with goblet cell hyperplasia in asthmatic airways (Takeyama et al., 2001, Am. J. Respir. Crit. Care Med., 163(2):511-516.). Any attempted repair of initial epithelial cell damage leads to hyperproliferation and differentiation responses that are linked to EGFR and EGFR activation (Bonner, 2002, Am. J. Physiol. Lung Cell Mol. Physiol., 283(3):L528-530). Asthmatics appear to develop chronically high levels of EGFR even in undamaged epithelium. This sustains a constant inflammatory condition, and leads to fibrosis and mucus hypersecretion associated with airway obstruction, morbidity and lethality in asthma, COPD, and other pulmonary diseases.

### UCHL1 and Parkinson's Disease

UCHL1, or ubiquitin carboxy terminal hydrolase, is genetically associated with Parkinson's Disease (PD) (Chung et al., 2003, J. Neurol., 250 Suppl. 3:11115-11124; Toda et al., 2003, J. Neurol., 250 Suppl. 3:11140-11143; Maraganore et al., 2004, Ann. Neurol., 55(4):512-521). Mutations in UCHL1 cause autosomal dominant PD, consistent with the notion that derangements in the ubiquitin proteasomal pathway play important roles in the demise of dopamine neurons in PD.

Other proteases/proteolytic enzymes are associated with other diseases, as is known in the art. Examples of proteases, including isopeptidases and other proteolytic enzymes associated with diseases or physiological conditions are as follows.
USP2a prostatic cancer
Ap-UCH essential for long-term memory in Aplysia
BAP1 tumor suppressor (associates with BRCA1)
CYLD1 tumor suppressor
DUB-1 cytokine-inducible, B-cell selective
DUB-2 cytokine-inducible, T-cell selective
D-ubp-64E Drosophila inhibitor of position-effect variegation
FAF (Fat facets) Drosophila eye development
FAM pre-implantation mouse embryo development
HAUSP (USP7) tumor suppressor (p53 stabilization)
USP10 p53 regulator; DNA damage
Tre-2 (USP6) oncoprotein
Ubp3 inhibitor of transcriptional silencing in yeast
UBP41 apoptosis, bone formation
UBP43 negative regulator of IFN signaling, hematopoesis
UBP45 myogenesis
UBP69 myogenesis
UbpB (Dictyostelium) developmental timing and spatial patterning
UBP-M (USP16) cell cycle control (chromatin condensation)
UBPY cell cycle/cell growth
USP14 (ataxia) synaptic function
UCH-L1 (PGP9.5) Parkinson's Disease, gracile axonal dystrophy
VDU1 (USP20) tumorigenesis (associates with von Hippel-Lindau protein)
VDU2 (USP33) tumorigenesis (associates with von Hippel-Lindau protein)
Caspases Alzheimer's disease, apoptosis, necrosis, inflammation, ischemia
Trypsins cystic fibrosis
Tryptases mastocytosis
Chymotrypsins pancreatitis
Cathepsins cancer, stroke, Alzheimer's disease, arthritis, Ebola virus infection, chronic obstructive pulmonary disorder (COPD), chronic periodontitis, keratoconus, retinal detachment, age-related macular degeneration, and glaucoma
Beta-secretases (BACE) Alzheimer's disease
Proconvertases/furins atherosclerosis, dyslipidemia, obesity, cancer

### Kits

In another aspect, the present invention provides kits for detecting protease activity. In particular embodiments, the present invention provides kits for detecting isopeptidase activity. In some embodiments, the kits comprise one or more protease substrates as described hereinabove. In a particular embodiment, the protease substrate comprises (i) a first moiety comprising ubiquitin (Ub) or a ubiquitin-like protein (Ubl), said first moiety comprising at its C-terminus a cleavage site for the protease, and (ii) a second moiety comprising a luciferase substrate, wherein the first moiety is covalently linked at its C-terminus to the second moiety via an amide linkage. In some embodiments, the kits comprise one or more isopeptidase substrates comprising (i) a first moiety comprising ubiquitin (Ub) or a ubiquitin-like protein (Ubl), and (ii) a second moiety comprising a luciferase substrate, wherein the first moiety is covalently linked at its C-terminus to the second moiety via an amide linkage. The kits may optionally comprise one or more luciferase enzymes, and may optionally comprise instructions. Luciferase enzymes optionally included in the kit include, but are not limited to luciferase from Photinus pyralis, Aequorea victoria, Renilla reniformis and Gaussia princeps.

In some embodiments, the luciferase substrate is luciferin or coelenterazine.

Optional instructions may explain how to conduct the assay, how to detect luminescence, and/or how to correlate luminescence to isopeptidase activity. Other optional reagents in the kit can include appropriate buffers for isopeptidase and luciferase activity.

As used herein, “instructions” or “instructional material” includes a publication, a recording, a diagram, or any other medium of expression which can be used to communicate the usefulness of the composition of the invention for performing a method of the invention. The instructions or instructional material of a kit of the invention can, for example, be affixed to a container which contains a kit of the invention to be shipped together with a container which contains the kit. Alternatively, the instructions or instructional material can be shipped separately from the container with the intention that the instructions or instructional material and kit be used cooperatively by the recipient.

The following examples are provided to illustrate various embodiments of the present invention. The examples are illustrative and are not intended to limit the invention in any way.

### EXAMPLE I

### Preparation of glycyl-D-aminoluciferin

A. Synthesis of 2-Cyano-6-amino-N-Boc-glycine-benzothiazole. Commercially available Boc-Gly-OH (675 mg, 3.85 mmol) was dissolved in 18 mL CH₂Cl₂. DIC (0.6 mL, 3.9 mmol) and DMAP (47 mg, 0.39 mmol) were added at 0° C. and stirred at 0° C. for 20 min. 2-Cyano-6-aminobenzothiazole (450 mg, 2.57 mmol) was added to the reaction mixture and the reaction was stirred at room temperature for 3 hours. The reaction was diluted with CH₂Cl₂ and washed with brine. The organic layer was dried over anhydrous sodium sulfate, filtered and concentrated. The crude product was purified by preparative reverse phase HPLC column using a linear gradient of 10-90% ACN over 10 min with a flow rate of 20 mL/min. After lyophilization, 2-Cyano-6-amino-N-Boc-glycine-benzothiazole was obtained as a white solid and characterized by LC-MS.

B. Synthesis of Boc-glycine-D-aminoluciferin. 2-Cyano-6-amino-N-Boc-glycine-benzothiazole (36 mg, 0.11 mmol) was dissolved in 0.7 mL of dimethylformamide (DMF); 21 mg of H-D-cysteine-HCl (dissolved in 0.1 mL of degassed water) was added drop-wise to the organic solution, while protected from light. The mixture was adjusted to pH 8.0 by addition of saturated K₂CO₃ in degassed water, and the reaction was stirred for 1 h at room temperature protected from light. Analytical HPLC and mass spectrometry analysis (ESI-MS) showed complete conversion to the product. The crude reaction mixture was purified by preparative reverse phase HPLC column using a linear gradient of 10-90% ACN over 10 min with a flow rate of 20 mL/min. After lyophilization, Boc-glycyl-D-aminoluciferin was obtained as a white solid and characterized by LC-MS.

C. Boc-glycyl-D-aminoluciferin was dissolved in 1:1 TFA: CH₂Cl₂ with 5% Et₃SiH and allowed to stir for 1.5 hours. The mixture was concentrated in a vacuum, and the residue was directly purified by preparative reverse phase HPLC column using a linear gradient of 0-45% ACN over 15 min with a flow rate of 20 mL/min. After lyophilization, pure glycyl-D-aminoluciferin was obtained as a light yellow solid and characterized to verify predicted molecular weight by LC-MS.

### EXAMPLE 2

### Preparation of Ub(1-76)-aminoluciferin

Recombinant fusion protein ubiquitin(1-75)-intein was produced in accordance with the protocol described in Hassiepen et al., 2007, Analyt. Biochem., 371:201-207. The ubiquitin(1-75)-intein fusion protein was immobilized on affinity resin and cleaved with mercaptoethanesulfonic acid (MESNa), to generate a thioester derivative (Ub-MESNa).

The Ub-MESNa was purified in accordance with Hemelarr et al. (2004, Mol. Cell. Biol., 24:84-95). Purified Ub/Ubl-MESNa was subjected to conjugation with glycyl-D-amino-luciferin using a modified version of published protocols (Wilkinson et al., 2005, supra). The specific modifications to the protocol resulted in optimal solubility of the luciferin molecule and optimal efficiency of the conjugation reaction. The specific modifications were to the solvent used (2:1 DMF/DMSO, plus 2% triethylamine) and the temperature (40° C.) and duration (16 hours) of the conjugation reaction.

Ub-MESNa and glycyl-D-aminoluciferin were mixed in a molar ratio of 1:100 in 2:1 DMF/DMSO, plus 2% triethylamine. The final concentration of Ub-MESNa was 1 mM. The reaction kinetics were monitored by LC-MS. After 16 hours (40° C.), the conjugation was complete; prolonged reaction time did not result in higher product yields. The crude reaction mixture was purified by preparative reverse phase HPLC column using a linear gradient of 20-50% ACN over 20 min (flow rate of 20 mL/min). After lyophilization, pure Ub(1-76)-aminoluciferin was obtained as a white solid and characterized by LC-MS (See FIG. 1).

### EXAMPLE 3

The Ub-amino-luciferin substrate was compared to the Z-Arg-Leu-Arg-Gly-Gly (SEQ ID NO: 1)-amino-luciferin substrate (Promega DUB-Glo™ substrate) to determine the limit of detection of the activities of four different isopeptidases (UCHL3, USP2core, USP7, and USP8). For all enzymes, these two substrates were tested in parallel using the DUB-Glo™ assay system (Promega) according to the manufacturer's instructions. All enzymes were diluted into 50 mM HEPES, pH 7.5, 10 mM DTT, 0.1% Prionex (Sigma-Aldrich). For testing Z-Arg-Leu-Arg-Gly-Gly (SEQ ID NO: 1)-amino-luciferin, final isopeptidase concentrations were as follows: UCHL3 equal to approximately 0.35 to 50 nM; USP2core equal to approximately 10 to 500 nM, USP7 equal to approximately 0.1 to 50 nM, and USP8 equal to approximately 20 to 500 nM. For testing Ub-amino-luciferin, final isopeptidase concentrations were as follows: UCHL3 equal to approximately 0.05 to 50 pM; USP2core, USP7, and USP8 equal to approximately 0.5 to 500 pM. Reactions were assembled through addition of 50 μl of diluted enzyme and 50 μl of DUB-Glo™ reaction mixture supplemented with either Z-Arg-Leu-Arg-Gly-Gly (SEQ ID NO: 1)-amino-luciferin (final concentration of 20 μM) or Ub-amino-luciferin (final concentration of 100 nM). The signal in relative luminescence units (RLUs) was measured at 30 minutes from reaction assembly on an Envision multi-label plate reader (Perkin Elmer). The signal to background ratio (S/B) was calculated as (maximum signal−signal in absence of enzyme)/(signal in absence of enzyme).

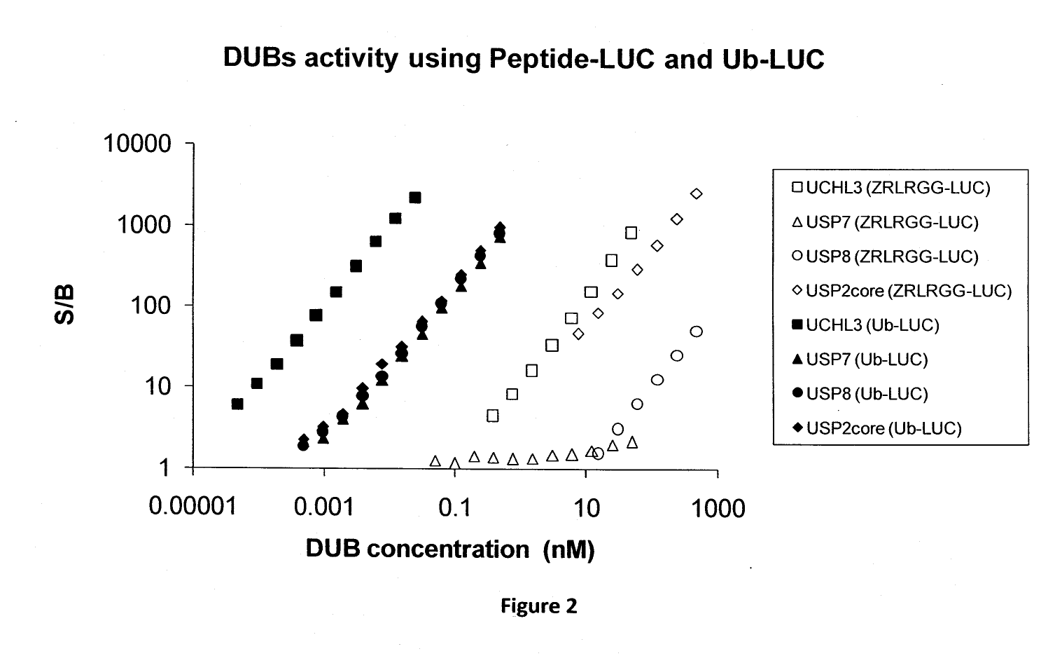

A linear UCHL3-dependent bioluminescence S/B was achieved with both substrates, however the linear range for the S/B using Ub-amino-luciferin established limits of detection approximately 1000-fold lower. Specifically, using Z-RLRGG(SEQ ID NO: 1)-amino-luciferin at a concentration of 20 μM and UCHL3 at a concentration of 40 nM, a signal to background (S/B) ratio of ˜1000 was obtained. Under identical reaction conditions, the same S/B ratio of 1000 was obtained with 100 nM Ub-amino-luciferin and UCHL3 at a concentration of 32 pM. A linear UCHL3-dependent bioluminescence signal can be achieved with both substrates, however the linear signal for Ub-luciferin establishes limits of detection approximately 1000-fold lower (see FIG. 2).

The Promega DUB-Glo™ assay utilizes a peptide substrate (Z-Arg-Leu-Arg-Gly-Gly; SEQ ID NO: 1) coupled to amino-luciferin, such that cleavage by a DUB after the C-terminal Gly liberates amino-luciferin, which in turn is consumed by luciferase to generate light. The sequence of this peptide is derived from the peptide sequence found at the C-terminus of ubiquitin. This substrate is a relatively poor substrate for DUBs, as judged by its affinity for the DUB enzymes in general, and that the assay requires its use at high molar concentrations (40 μM). In addition, this substrate does not exhibit a high degree of specificity for DUBs over other Ubl-specific proteases (e.g., deSUMOylases, deNEDDylases, etc.). Z-Arg-Leu-Arg-Gly-Gly (SEQ ID NO: 1)-amino-luciferin is reported to react with comparable efficiency with members of every class of Ub/Ubl-specific protease. This is likely due, at least in part, to differences in the mechanism between a short peptide substrate and the biologically relevant protein substrate (in this case the particular Ub/Ubl protein). Peptide substrates are largely characterized as being recognized by the enzyme by local interactions in and around the active site of the enzyme. In stark contrast, Ub/Ubls are widely thought to bind to their cognate enzymes through extended interactions away from the active site, which may include multiple shared contact points between the enzyme and the substrate. These “tertiary interactions” not only facilitate enhanced catalytic efficiency, but likely play a role in determining specificity for Ub/Ubl cleavage. The current technology, therefore, provides a very different luciferase-based platform. While DUB-Glo™ technology results in poor cleavage efficiency and indiscriminant cleavage, the current technology benefits both from a high degree of enzyme specificity (for example, a Ub-luciferin substrate will not cross-react with deSUMOylases) and enhanced catalytic efficiency.

### EXAMPLE 4

### Comparison of Ub-amino-luciferin to Ub-AMC and Ub-rhodamine

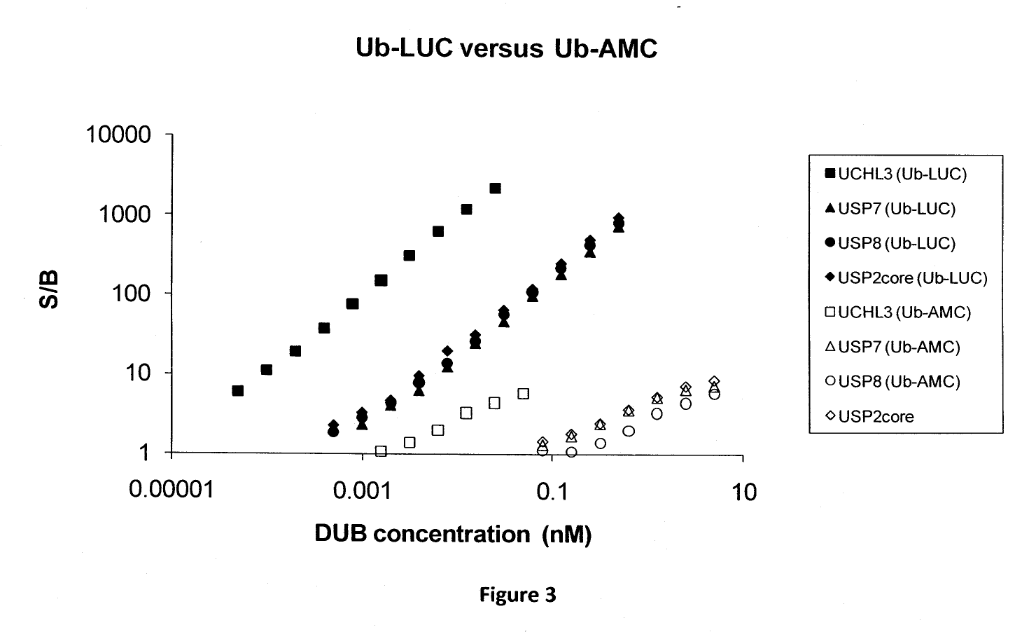

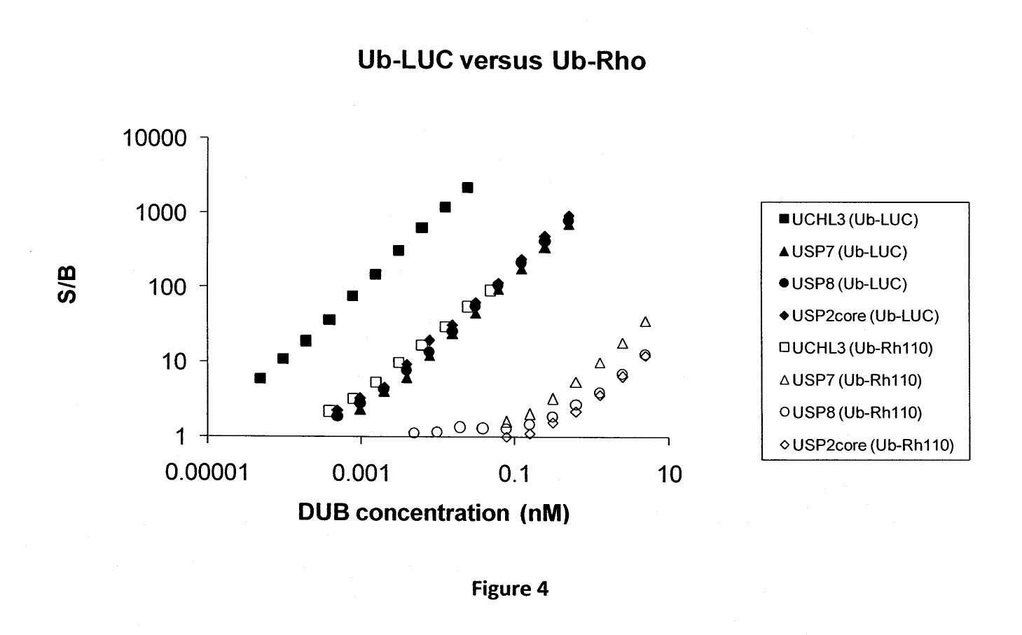

The Ub-amino-luciferin (Ub-LUC) substrate was compared to the Ub-AMC substrate (FIG. 3) and the Ub-Rhodamine110 (Ub-Rho) substrate (FIG. 4), to determine the limit of detection for the activities of four different isopeptidases (UCHL3, USP2core, USP7, and USP8). All enzymes were diluted into 50 mM Tris, pH 8.0, 5 mM DTT, 0.05% (w/v) CHAPS. Final isopeptidase concentrations were as follows: UCHL3 equal to approximately 2 pM to 40 pM; USP2core, USP7, and USP8 were equal to approximately 10 to 1000 nM. Either Ub-AMC or Ub-Rhodamine110 (final concentration equal to 250 nM) were diluted into 50 mM Tris, pH 8.0, 5 mM DTT, 0.05% (w/v) CHAPS. Reactions were assembled through the addition of 50 μl diluted isopeptidase to 50 μl of either Ub-AMC or Ub-Rhodamine110, and the signal in relative fluorescence units (RFUs) was measured at 30 minutes on an Envision multi-label plate reader (Perkin Elmer). For Ub-AMC, fluorescence was monitored with filters corresponding to excitation at 340 nm and emission at 460 nm. For Ub-Rhodamine110, fluorescence was monitored with filters corresponding to excitation at 485 nm and emission at 531 nm. The signal to background (S/B) was calculated as (maximum signal−signal in absence of enzyme)/(signal in absence of enzyme). Using the Ub-amino-luciferin substrate, the limit of detection (typically defined as yielding a S/B>3) for these enzymes (UCHL3, USP2core, USP7, and USP8) was increased by a factor of about 60-fold to about 120-fold over Ub-AMC (see FIG. 3), and by more than 30-fold over Ub-rhodamine110 (see FIG. 4).

The Ub/Ubl-AMC format (and the analogous format employing the alternate fluorophore is limited in 1) achieving maximal signal to background (S/B) by intrinsic fluorescence of the starting conjugate, and 2) ability to discover small organic molecule inhibitors of isopeptidases due to the potential assay interference often ascribed to the spectral properties of said inhibitors (e.g., auto-fluorescence, quenching, etc.). As stated above, the high substrate specificity that luciferase has for free luciferin, coupled with the mechanism of signal generation in this assay, greatly improves S/B and minimizes spectral artifact associated with fluorogenic-based Ub/Ubl assays. We have assessed the S/B (calculated as maximum signal−signal in absence of enzyme/signal in absence of enzyme) for Ub-amino-luciferin in comparison to both Ub-AMC and Ub-Rhodamine110. All three ubiquitin derivative substrates were tested at comparable molar concentrations. For the DUB UCHL3 (0.01 nM) the S/B for Ub-amino-luciferin is ˜50-fold greater than the S/B for Ub-AMC and ˜200-fold greater than the S/B for Ub-rhodamine110. Furthermore, the S/B for Ub-amino-luciferin with the enzymes USP7, USP8, and the core domain of USP2 (USP2core) (all assayed at 0.5 nM) was increased by ˜100-fold relative to the S/B for both Ub-AMC and Ub-Rhodamine110. These data indicate that Ub-amino-luciferin has a greater sensitivity towards these enzymes, resulting in an increased lower limit of detection. In fact the limit of detection (typically defined as yielding a S/B>3) using Ub-amino-luciferin with these enzymes was increased by a factor of about 60-fold to about 120-fold over the Ub-AMC substrate, and was increased by more than 30-fold over Ub-rhodamine110 substrate.

It is known in the art some DUBs exhibit activity in the form of polyubiquitin chain degradation, or removal of ubiquitin (in mono- or poly-form) from naturally occurring (target protein) substrates, yet do not yield detectable activity towards Ub-AMC or Ub-rhodamine110. The catalytic mechanism by which a DUB cleaves Ub-luciferin is likely to be very similar to Ub-AMC or Ub-rhodamine110, as these molecules are all composed of a full-length ubiquitin molecule with a small adjunct reporter molecule attached to the C-terminus. Therefore, it would be reasonable to assume that DUBs not yielding detectable activity towards Ub-AMC or Ub-rhodamine110 also would not yield detectable activity towards Ub-luciferin. Surprisingly, however, a number of DUBs that do not yield detectable activity towards Ub-AMC and Ub-rhodamine110 (as judged by having a S/B>3), do in fact yield significantly higher S/B ratios with Ub-luciferin. As detailed in Table 1 (see below Example 5), a number of DUBs were assayed with Ub-amino-luciferin, Ub-AMC, and Ub-Rhodamine110 at equivalent molar concentrations, and approximately 10-1000 fold higher S/B was associated with Ub-amino-luciferin, with no detectable activity towards either Ub-AMC or Ub-Rhodamine110.

### EXAMPLE 5

The Ub-amino-luciferin substrate was compared to 1) Ub-AMC 2) Ub-Rhodamine110, and 3) Z-Arg-Leu-Arg-Gly-Gly (SEQ ID NO: 1)-luciferin to determine the limit of detection of the activities of nine different isopeptidases (Otub2, JosD1, JosD2, AMSH, Ataxin3, Ataxin3-like, UCHL5, USP20, and USP14). For Ub-amino-luciferin and Z-Arg-Leu-Arg-Gly-Gly (SEQ ID NO: 1)-luciferin, all enzymes were diluted into 50 mM HEPES, pH 7.5, 10 mM DTT, 0.1% Prionex (Sigma-Aldrich) to a final concentration of 10 nM. For Ub-AMC and Ub-Rhodamine110, all enzymes were diluted into 50 mM Tris, pH 8.0, 5 mM DTT, 0.05% CHAPS to a final concentration of 10 nM. Either Ub-amino-luciferin or Z-Arg-Leu-Arg-Gly-Gly (SEQ ID NO: 1)-luciferin was diluted into DUB-Glo™ reagent for final reaction concentrations of 100 nM or 20 μM, respectively. Either Ub-AMC or Ub-Rhodamine110 (final reaction concentration equal to 250 nM) were diluted into 50 mM Tris, pH 8.0, 5 mM DTT, 0.05% (w/v) CHAPS. Reactions were assembled through the addition of 50 μl diluted isopeptidase to 50 μl of Ub-AMC, Ub-Rhodamine110, Ub-amino-luciferin, or Z-Arg-Leu-Arg-Gly-Gly (SEQ ID NO: 1)-luciferin and the signal was measured at 30 minutes. For Ub-amino-luciferin and Z-Arg-Leu-Arg-Gly-Gly (SEQ ID NO: 1)-luciferin, luminescence (RLUs) was measured, while for Ub-AMC, fluorescence was monitored with filters corresponding to excitation at 340 nm and emission at 460 nm. For Ub-Rhodamine110, fluorescence was monitored with filters corresponding to excitation at 485 nm and emission at 531 nm. The signal to background ratio was calculated as (maximum signal−signal in absence of enzyme)/(signal in absence of enzyme).

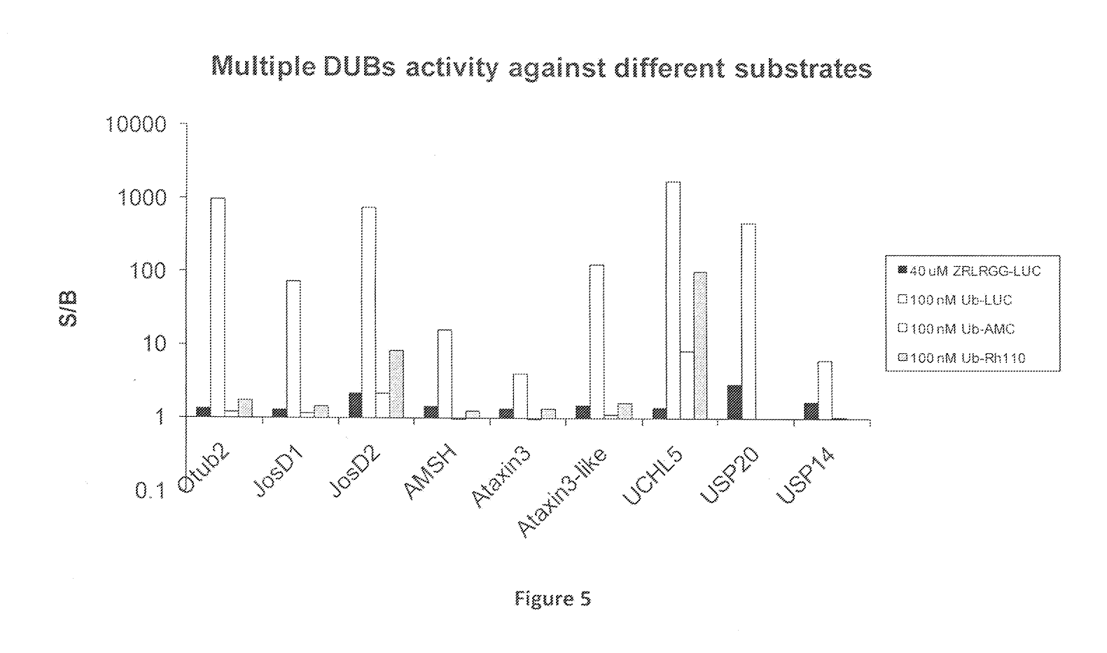

For all enzymes tested, Ub-amino-luciferin was shown to be a superior luciferase substrate reagent, detecting activity equal to 10-1000 fold (S/B) higher than the DUB-Glo™ reagent, Z-Arg-Leu-Arg-Gly-Gly (SEQ ID NO: 1)-luciferin. Most surprising, however, was that this comparative result demonstrated that seven of nine DUBs tested also display a S/B of approximately 10-1000-fold towards Ub-amino-luciferin, with little to no detectable activity towards Ub-AMC or Ub-rhodamine110. The exceptions in this case were JosD2 and UCHL5 (see FIG. 5 and Table 1).

TABLE 1
Platform comparison for various DUBs.

| | | | | | | Ataxin3- | | | |
|---|---|---|---|---|---|---|---|---|---|
| | Otub2 | JosD1 | JosD2 | AMSH | Ataxin3 | like | UCHL5 | USP20 | USP14 |
| Ub- | 1.2 | 1.1 | 2.1 | 1.0 | 0.9 | 1.1 | 8.1 | ND | 1.0 |
| AMC | | | | | | | | | |
| Ub- | 1.7 | 1.5 | 8.2 | 1.2 | 1.3 | 1.6 | 103 | ND | ND |
| rho | | | | | | | | | |
| DUB- | 1.3 | 1.3 | 2.1 | 1.4 | 1.3 | 1.5 | 1.4 | 2.8 | 1.6 |
| glo ™ | | | | | | | | | |
| Ub- | 980 | 74 | 760 | 16 | 4.0 | 120 | 1700 | 473 | 6.0 |
| LUC | | | | | | | | | |

Signal to Background values [(maximum signal − background signal)/background signal] are provided. The background signal is measured in the absence of the DUB.
The DUB was present at a concentration of 10 nM.
ND = not determined.

### EXAMPLE 6

The hSUMO2-amino-luciferin substrate was compared to the Z-Arg-Leu-Arg-Gly-Gly (SEQ ID NO: 1)-amino-luciferin substrate (Promega DUB-Glo™ substrate) to determine the specificity and limit of detection of the activities of six (6) different isopeptidases (USP34, USP7, USP8, SENP1, SENP2, and SENP6). For all enzymes, these two substrates were tested in parallel using the DUB-Glo™ assay system (Promega) according to the manufacturer's instructions. All enzymes were diluted into 50 mM HEPES, pH 7.5, 10 mM DTT, 0.1% Prionex® (Sigma-Aldrich). For testing Z-Arg-Leu-Arg-Gly-Gly (SEQ ID NO: 1)-amino-luciferin, final isopeptidase concentrations for all enzymes were equal to approximately 70 pM to 50 nM. For testing hSUMO2-amino-luciferin, final isopeptidase concentrations for all enzymes were equal to approximately 70 pM to 50 nM. Reactions were assembled in a 384-well plate through addition of 15 μl of diluted enzyme and 15 μl of DUB-Glo™ reaction mixture supplemented with either Z-Arg-Leu-Arg-Gly-Gly (SEQ ID NO: 1)-amino-luciferin (final concentration of 20 μM) or hSUMO2-amino-luciferin (final concentration of 100 nM). The signal in relative luminescence units (RLUs) was measured at 30 minutes from reaction assembly on an EnVision™ multi-label plate reader (Perkin Elmer). The signal to background ratio (S/B) was calculated as (maximum signal/(signal in absence of enzyme) plus or minus the corrected standard deviation of triplicate measurements.

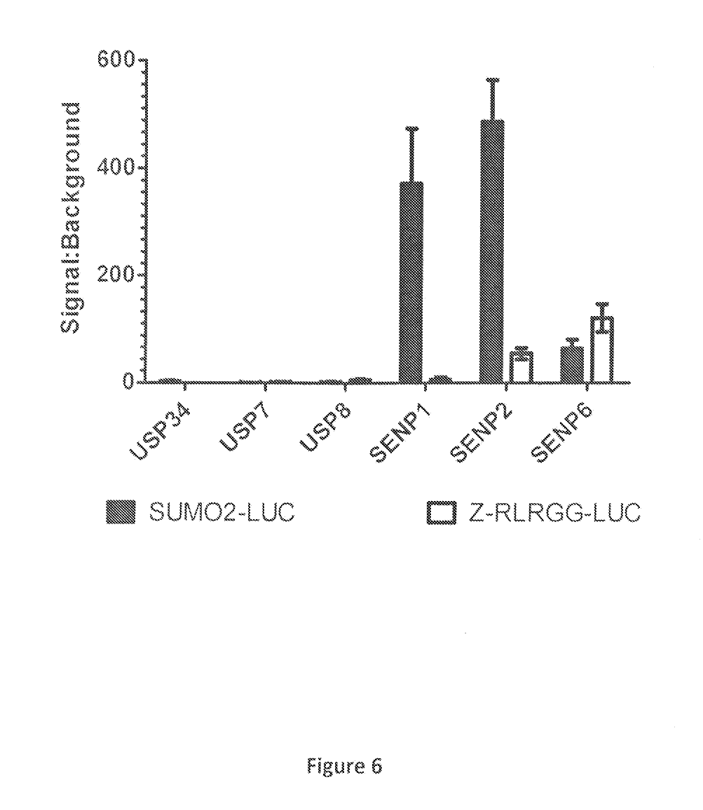

As expected, hSUMO2-amino-luciferin yielded virtually no signal with the DUBs USP34, USP7, and USP8 at concentrations equal to 200 pM (FIG. 6). These enzymes are ubiquitin specific and do not recognize nor cleave SUMO-based substrates. SENP1, SENP2, and SENP6 (200 pM) on the other hand gave strong signals with hSUMO2-amino-luciferin (FIG. 6). Interestingly, Z-Arg-Leu-Arg-Gly-Gly (SEQ ID NO: 1)-amino-luciferin also gave signals with SENP2 and SENP6 (200 pM). Since this sequence is quite different from the C-terminal sequence of hSUMOs, i.e. Gln-Gln-Thr-Gly-Gly (SEQ ID NO: 30), these results demonstrate the lack of specificity of the Z-RLRGG (SEQ ID NO: 1)-amino-luciferin substrate.

This substrate is a relatively poor substrate for DUBs, as judged by its affinity for the DUB enzymes in general, and that the assay requires its use at high molar concentrations (20 μM). Z-Arg-Leu-Arg-Gly-Gly (SEQ ID NO: 1)-amino-luciferin is reported to react with comparable efficiency with members of every class of Ub/Ubl-specific protease. This is likely due, at least in part, to differences in the mechanism between a short peptide substrate and the biologically relevant protein substrate (in this case the particular Ub/Ubl protein). Peptide substrates are largely characterized as being recognized by the enzyme by local interactions in and around the active site of the enzyme. In stark contrast, Ub/Ubls are widely thought to bind to their cognate enzymes through extended interactions away from the active site, which may include multiple shared contact points between the enzyme and the substrate. These “tertiary interactions” not only facilitate enhanced catalytic efficiency, but likely play a role in determining specificity for Ub/Ubl cleavage. The current technology, therefore, provides a very different luciferase-based platform. While DUB-Glo™ technology results in poor cleavage efficiency and indiscriminant cleavage, the current technology benefits both from a high degree of enzyme specificity (for example, a Ub-luciferin substrate will not cross-react with deSUMOylases) and enhanced catalytic efficiency.

### EXAMPLE 7

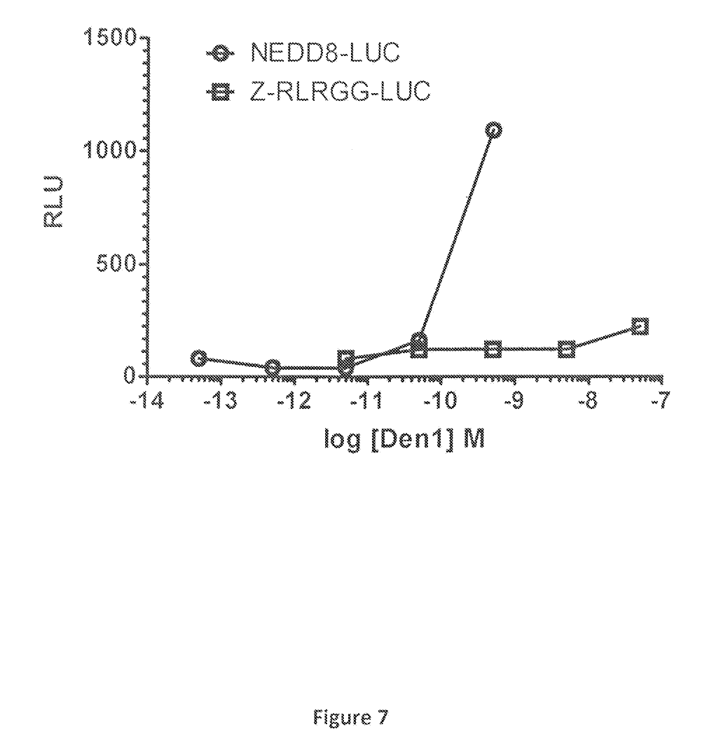

The NEDD8-amino-luciferin substrate was compared to the Z-Arg-Leu-Arg-Gly-Gly-(SEQ ID NO: 1) amino-luciferin substrate (Promega DUB-Glo™ substrate) to determine the limit of detection of the activities of the isopeptidase DEN1. The two substrates were tested in parallel using the DUB-Glo™ assay system (Promega) according to the manufacturer's instructions. The enzyme was diluted into 50 mM HEPES, pH 7.5, 10 mM DTT, 0.1% Prionex® (Sigma-Aldrich) over a concentration range from approximately 50 fM to 1 nM for NEDD8-amino-luciferin and from 50 pM to 50 μM for Z-RLRGG (SEQ ID NO: 1)-amino-luciferin. Reactions were assembled in a 384-well plate through addition of 15 μl of diluted enzyme and 15 μl of DUB-Glo™ reaction mixture supplemented with either Z-Arg-Leu-Arg-Gly-Gly (SEQ ID NO: 1)-amino-luciferin (final concentration of 20 μM) or NEDD8-amino-luciferin (final concentration of 250 nM). The signal in relative luminescence units (RLUs) was measured at 30 minutes from reaction assembly on an EnVision™ multi-label plate reader (Perkin Elmer). A significant increase in signal was observed with the NEDD8-amino-luciferin substrate at a Den1 concentration at least 3-orders of magnitude lower than required for the Z-RLRGG (SEQ ID NO: 1)-amino-luciferin substrate (FIG. 7).

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for detecting the activity of a protease in a sample comprising
(A) contacting said sample with
(i) a protease substrate comprising
(a) a first moiety comprising at least one ubiquitin (Ub) or a ubiquitin-like protein (Ubl), said first moiety comprising at its C-terminus a cleavage site for said protease, and
(b) a second moiety comprising a luciferase substrate,
wherein said first moiety is covalently linked at its C-terminus to said second moiety via an amide linkage; and
(ii) luciferase,
wherein said protease cleaves said protease substrate at the C-terminal end of the first moiety, thereby generating a free luciferase substrate; and
(B) detecting luminescence in said sample, wherein luminescence is indicative of the activity of said protease.

2. The method of claim 1, wherein said protease is selected from the group consisting of deubiquitinating enzyme, Ubl-specific protease (Ulp), isopeptidase, caspase, trypsin, tryptase, cathepsin, chymotrypsin, and β-secretase.

3. The method of claim 2, wherein said protease is a deubiquitinating enzyme or Ulp.

4. The method of claim 1, wherein the luminescence has a signal to background ratio (S/B) about 10- to about 1000-fold greater than the S/B for a corresponding assay wherein the first moiety is a C-terminal peptide of Ub or a Ubl or the second moiety is a fluorophore.

5. The method of claim 1, wherein the luminescence has a signal to background ratio (S/B) about 10- to about 1000-fold greater than the S/B for a corresponding assay wherein the first moiety is a C-terminal peptide of Ub or a Ubl and the second moiety is a fluorophore.

6. The method of claim 1, wherein the luciferase substrate is luciferin or coelenterazine.

7. The method of claim 6, wherein the luciferase substrate is coelenterazine and the luciferase is from an aquatic species selected from the group consisting of Aequorea victoria, Renilla renifirmis and Gaussia princeps.

8. The method of claim 6, wherein the luciferase substrate is luciferin and the luciferase is from Photinus pyralis.

9. The method of claim 3, wherein said deubiquitinating enzyme or a ubiquitin-like protein (Ubl)-specific protease (Ulp) is selected from the group consisting of UCHL3, USP2core, USP7, USP8, USP34, Otub2, JosD1, JosD2, AMSH, Ataxin3, Ataxin3-like, UCHL5, USP20, USP14, ULP1, Ulp2, SENP1, SENP2, SENP6, SENP8, A20 and SENP5.

10. The method of claim 1, wherein the sample is selected from the group consisting of a cellular lysate, a cellular extract, a reaction mixture, or a bodily fluid.

11. The method of claim 1, wherein the Ubl is selected from the group consisting of small ubiquitin like-modifier-1 (SUMO), SUMO-2, SUMO-3, ISG-15, NEDD-8, ISG-15, APG12, URM1, and APG8.

12. A protease substrate comprising
(A) a first moiety comprising at least one ubiquitin (Ub) or a ubiquitin-like protein (Ubl), said first moiety comprising at its C-terminus a cleavage site for a protease; and
(B) a second moiety comprising a luciferase substrate,
wherein the first moiety is covalently linked at its C-terminus to the second moiety via an amide linkage.

13. The protease substrate of claim 12, wherein said cleavage site is a deubiquitinating enzyme or Ubl-specific protease (Ulp) cleavage site.

14. A method for screening for agents capable of modulating the activity of a protease comprising
(A) contacting the protease with
(i) the protease substrate of claim 12,
(ii) at least one test agent, and
(iii) luciferase,
wherein the protease cleaves the protease substrate at the C-terminal end of the first moiety, thereby generating free luciferase substrate, and
(B) detecting luminescence in the sample, wherein luminescence is indicative of the protease activity,
wherein a difference in the level of luminescence in the presence of the test agent as compared to the absence of the test agent indicates that the agent modulates the activity of the protease.

15. A method for detecting an increased risk for a disease or condition associated with a protease in a subject, said method comprising
(A) contacting a sample obtained from said subject with
(i) the protease substrate of claim 12, and
(ii) luciferase,
wherein the protease cleaves the protease substrate at the C-terminal end of the first moiety, thereby generating free luciferase substrate, and
(B) detecting luminescence in the sample, wherein luminescence is indicative of the protease activity and indicates an increased risk for said disease or condition.

16. A method for identifying the cleavage site of a protease comprising
(A) contacting said protease with
(i) at least one protease substrate comprising
(a) a first moiety comprising at least one ubiquitin (Ub) or a ubiquitin-like protein (Ubl), said first moiety comprising at its C-terminus an amino acid sequence of interest, and
(b) a second moiety comprising a luciferase substrate,
wherein said first moiety is covalently linked at its C-terminus to said second moiety via an amide linkage; and
(ii) luciferase,
wherein said protease cleaves said protease substrate at the C-terminal end of the first moiety, thereby generating a free luciferase substrate; and
(B) detecting luminescence in said sample, wherein the presence of luminescence is indicative that the amino acid sequence of interest comprises said cleavage site.

17. A kit for detecting protease activity, comprising
(A) the protease substrate of claim 12,
(B) optionally, a luciferase, and
(C) optionally, instructions.

18. The kit of claim 17, wherein the luciferase substrate of said protease substrate is luciferin or coelenterazine.


SEQUENCE LISTING (30 sequences)

SEQ ID NO: 1 - protein, 5 aa, Artificial Sequence
  Synthetic Sequence
RLRGG

SEQ ID NO: 2 - protein, 76 aa, Homo sapiens [sequence omitted]

SEQ ID NO: 3 - protein, 4 aa, Artificial Sequence
  Synthetic Sequence
DEVD

SEQ ID NO: 4 - protein, 4 aa, Artificial Sequence
  Synthetic Sequence
YVAD

SEQ ID NO: 5 - protein, 4 aa, Artificial Sequence
  Synthetic Sequence
WEHD

SEQ ID NO: 6 - protein, 5 aa, Artificial Sequence
  Synthetic Sequence
VDVAD

SEQ ID NO: 7 - protein, 4 aa, Artificial Sequence
  Synthetic Sequence
LEHD

SEQ ID NO: 8 - protein, 4 aa, Artificial Sequence
  Synthetic Sequence
VEID

SEQ ID NO: 9 - protein, 4 aa, Artificial Sequence
  Synthetic Sequence
VEVD

SEQ ID NO: 10 - protein, 4 aa, Artificial Sequence
  Synthetic Sequence
VEHD

SEQ ID NO: 11 - protein, 4 aa, Artificial Sequence
  Synthetic Sequence
IETD

SEQ ID NO: 12 - protein, 4 aa, Artificial Sequence
  Synthetic Sequence
AEVD

SEQ ID NO: 13 - protein, 4 aa, Artificial Sequence
  Synthetic Sequence
LEXD

SEQ ID NO: 14 - protein, 4 aa, Artificial Sequence
  Synthetic Sequence
VEXD

SEQ ID NO: 15 - protein, 4 aa, Artificial Sequence
  Synthetic Sequence
IEHD

SEQ ID NO: 16 - protein, 4 aa, Artificial Sequence
  Synthetic Sequence
PEHD

SEQ ID NO: 17 - protein, 4 aa, Artificial Sequence
  Synthetic Sequence
XEHD

SEQ ID NO: 18 - protein, 4 aa, Artificial Sequence
  Synthetic Sequence
DEHD

SEQ ID NO: 19 - protein, 4 aa, Artificial Sequence
  Synthetic Sequence
LETD

SEQ ID NO: 20 - protein, 4 aa, Artificial Sequence
  Synthetic Sequence
XEXD

SEQ ID NO: 21 - protein, 4 aa, Artificial Sequence
  Synthetic Sequence
XXXD

SEQ ID NO: 22 - protein, 6 aa, Artificial Sequence
  Synthetic Sequence
XXXXXX

SEQ ID NO: 23 - protein, 6 aa, Artificial Sequence
  Synthetic Sequence
XXXXEX

SEQ ID NO: 24 - protein, 6 aa, Artificial Sequence
  Synthetic Sequence
XXXXFR

SEQ ID NO: 25 - protein, 6 aa, Artificial Sequence
  Synthetic Sequence
XXXXFR

SEQ ID NO: 26 - protein, 6 aa, Artificial Sequence
  Synthetic Sequence
XXXXRR

SEQ ID NO: 27 - protein, 6 aa, Artificial Sequence
  Synthetic Sequence
XXXXRR

SEQ ID NO: 28 - protein, 5 aa, Artificial Sequence
  Synthetic Sequence
VNLDA

SEQ ID NO: 29 - protein, 4 aa, Artificial Sequence
  Synthetic Sequence
LRGG

SEQ ID NO: 30 - protein, 5 aa, Artificial Sequence
  Synthetic Sequence
QQTGG